SEQUENCE LISTING (2 sequences)

SEQ ID NO: 1 - protein, 14 aa, Homo sapiens
  Isoaspartyl (7)..(7): Isoaspartyl-Asn or Isoaspartyl-Asp
CTSNTSXGPSSNNR

SEQ ID NO: 2 - protein, 13 aa, Homo sapiens
CNNTANGPTTINN

(19) United States
(12) Patent Application Publication
(10) Pub. No.: US 2015/0232539 A1
(43) Pub. Date: Aug. 20, 2015

(54) Methods and Compositions for Detecting, Imaging, and Treating Small Cell Lung Cancer Utilizing Post-Translationally Modified Residues and Higher Molecular Weight Antigenic Complexes in Proteins

(71) Applicant: UNIVERSITY OF SOUTHERN CALIFORNIA, Los Angeles, CA (US)

(72) Inventors: Ite A. Laird, South Pasadena, CA (US); Meleeneh K. Derhartunian, San Mateo, CA (US); Mario A. Pulido, La Habra, CA (US); Dana W. Aswad, Irvine, CA (US)

(73) Assignee: UNIVERSITY OF SOUTHERN CALIFORNIA, Los Angeles, CA (US)

(21) Appl. No.: 14/426,122

(22) PCT Filed: Sep. 5, 2013
(86) PCT No.: PCT/US13/58344

§ 371 Date: Mar. 4, 2015

Related U.S. Application Data
(60) Provisional application No. 61/697,165, filed on Sep. 5, 2012.

(51) Int. Cl. C07K 16/18

(52) U.S. Cl. CPC C07K 16/18; C07K 2317/24; A61K 2039/505

(57) ABSTRACT

The present invention discloses ectopically expressed isoaspartylated proteins and antigenic peptide fragments thereof as potential biomarkers for cancer such as small cell lung cancer. Also disclosed are antigen recognition agents capable of specifically recognizing and binding to isoaspartylated proteins and/or antigenic peptide fragments thereof. Antigen recognition agents of the invention may be formed from proteins, antibodies, RNA aptamers, or other small molecules capable of specifically binding to an isoaspartylated protein or an antigenic peptide fragment thereof. Other aspects disclosed herein include imaging methods for using the isoaspartyl antigen recognition agents, therapeutic methods for treating small cell lung cancer and compositions for performing the same.

### CROSS-REFERENCE TO RELATED APPLICATIONS

This application claims the benefit of Provisional Application No. 61/697,165 filed on Sep. 5, 2012. The above priority application is hereby incorporated herein by reference.

### STATEMENT REGARDING FEDERALLY SPONSORED RESEARCH OR DEVELOPMENT

This invention was made, in part, with government support under DOD Concept Award W81XWH-10-0622 awarded by the Department of Defense, and, in part, by the Norris Comprehensive Cancer Center Core Grant P30 CA014089. The government has certain rights in the invention.

### SEQUENCE LISTING

This application contains sequence listing.

### FIELD OF THE INVENTION

The present invention relates generally to the detection, imaging and treatment of small cell lung cancer. More particularly, the present invention relates to the use of post-translationally modified residues and higher molecular weight antigenic complexes in proteins as biomarkers for the detection, imaging, and treatment of small cell lung cancer, including immunotherapy.

### BACKGROUND OF THE INVENTION

Lung cancer is one of the most prevalent forms of cancer. It can affect any part of the lung and is a leading cause of death in both men and women worldwide, including in the United States, Canada and China.

There are two types of lung cancer: Small-cell lung cancer (SCLC, also known as oat cell cancer) and non-small cell lung cancer (NSCLC). SCLC accounts for approximately 20% of all cases of lung cancer (1).

SCLC is the most aggressive lung cancer subtype. It grows rapidly, spreads quickly and is frequently associated with distinct paraneoplastic syndromes (a collection of symptoms that result from substances produced by the tumor). The good news is that SCLC responds well to chemotherapy and radiation. The bad news is that even when patients respond to chemotherapy treatment, the cancer quickly comes back; only 5% of the patients are alive after 5 years. It is also very difficult to detect lung cancer early. Most people with early lung cancer do not have any symptoms, so only a small number of lung cancers are found at an early stage. While studies based on low-dose spiral CT (LDSCT) scan have shown that early detection can reduce the mortality rate up to 20%, the technique is not suitable for screening of all persons. It has only been approved for subjects age 55 and older with at least a 30-pack year history of smoking (which means a pack a day for 30 years, or equivalent, such as 2 packs per day for 15 years). In addition, the test is based on physical images which usually identify a host of structural features that turn out not to be cancer. In fact, only 4% of the suspicion lesion seen on LDSCT are actually cancer. In other words, 96% are “false positives”. To make a definitive diagnosis, follow-up tests are required. Another potential screening test, sputum cytology, has been shown to be ineffective in reducing mortality rate presumably because the cancer found by sputum is not at an early enough stage to make a difference. In other forms of cancer, routine screening has made a significant difference in mortality rate. While LDSCT has recently been recommended for screening of long time smokers age 55 and over (70), no screening is available for people who do not match these criteria.

Thus, there currently exists an urgent need for a method to screen, detect and treat SCLC early.

### SUMMARY OF THE INVENTION

In view of the above, one object of the present invention is to provide a method for screening and/or early detection of SCLC. It is also one object of the present invention to provide a method for treating SCLC. It is still another object of the invention to provide compositions and reagents for performing screening, detection, and imaging methods of the present invention.

These and other objects of the present invention are satisfied, in part, by the unexpected discovery that SCLC cells will express post-translationally modified antigenic proteins. In particular, it is an unexpected discovery of the present invention that certain proteins (e.g. HuD—also known as ELAVL4, HuB, HuC, and by inference other SCLC-associated antigens such as the SOX family of proteins, Nova-1 etc.) carrying isoaspartate residues are expressed in SCLC cells and may serve as a biomarker for identifying SCLC. Moreover, it is also a discovery of the present invention that in addition to SCLC, other types of cancer cells such as neuroblastoma may also exhibit ectopic expression of neuronal proteins, the isoaspartylation of which may in turn serve as biomarkers for these cancer cells as well.

Accordingly, one aspect of the present invention is directed to a method of forming an isoaspartyl antigen recognition agent that is capable of specifically recognizing and binding to an isoaspartylated protein or an antigenic fragment thereof. Such antigen recognition agents may be useful for a number of applications including, but not limited to diagnostic imaging reagents, immunotherapeutic agent, or research tools. Methods in accordance with this aspect of the invention will generally include the steps of selecting a target protein; iso-aspartylating the protein or a peptide fragment thereof; and developing or identifying a molecule that is capable of specifically binding to the isoaspartylated protein or fragments thereof to serve as the antigen recognition agent.

In another aspect, the present invention is directed to an antigen recognition agent capable of specifically recognizing and binding to an isoaspartylated protein or an antigenic peptide fragment thereof.

In another aspect, the present invention provides a method for detecting SCLC cells. Methods in accordance with this aspect of the invention will generally include the steps of contacting a sample cell with an antigen recognition agent that specifically recognizes an isoaspartylated protein or an antigenic peptide fragment thereof.

In another aspect, the present invention also provides an imaging reagent for imaging the distribution of isoaspartylated proteins and antigenic peptide fragments thereof. Imaging reagents in accordance with this aspect of the invention will generally include an antigen recognition element operably linked to a signaling element.

In another aspect, the present invention provides a method for imaging the distribution of proteins containing isoaspartate residues. Methods in accordance with this aspect of the invention will generally include the steps of introducing an imagining reagent as described above into a test sample; allowing the antigen recognition agent to bind to a target antigen; and then detecting location of the imaging reagent.

In still another aspect, the present invention provides a therapeutic agent for treating SCLC. Therapeutic agents in accordance with this aspect of the invention will generally include an antigen recognition element specific for a isoaspartylated protein or an antigenic fragment thereof.

In yet another aspect, the present invention provides an immunotherapy treatment method for treating SCLC. Methods in accordance with this aspect of the invention will generally include the steps of administering an effective amount of an immune-therapeutic agent that contains isoaspartylated protein or antigenic fragments thereof, including aggregates of the protein that accumulate under isoaspartylation conditions, this agent would be administered as described above to a patient who has been determined as suffering from SCLC.

The materials, methods, and examples disclosed herein are illustrative only and not intended to be limiting. Other features, objects, and advantages of the invention will be apparent from the description and the accompanying drawings, and from the appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the mechanism of spontaneous isoaspartate formation and enzymatic repair in proteins. The formation of isoaspartyl sites (dashed arrows) occurs by spontaneous dehydration of an Asp-Xaa linkage or deamidation of an Asn-Xaa linkage to produce a metastable succinimide intermediate. The succinimide undergoes spontaneous hydrolysis to generate an unequal mixture of two products: typically, 15-30% of the normal L-aspartyl linkage (left solid arrow), with the remaining 70-85% forming an abnormal L-isoaspartyl-Xaa linkage (right dashed arrow). The isoaspartate repair pathway is indicated by the solid arrows. PIMT (also called PCMT1) converts L-isoaspartyl sites to α-carboxyl-O-methyl esters (downward pointing solid arrow). At physiological pH and temperature, the α-carboxyl-O-methyl esters have a typical half-life of only a few minutes and undergo spontaneous demethylation to reform the succinimide intermediate. The succinimide spontaneously hydrolyzes to generate either a normal L-aspartyl site or an L-isoaspartyl site. This inefficient repair process continues until the isoaspartyl residues have been repaired.

FIG. 2 shows the N-terminal region of HuD containing RNA Recognition Motif 1 (RRM1) and its upstream region, and that this protein section is prone to isoaspartyl conversion in vitro. A) Diagram of the N-terminal HuD sequence showing potential isoaspartyl sites (N or D) in bold, with boxes around canonical sites (N or D followed by a small amino acid such as G, S or H) and the RRM1 domain (dashed box). The 13-amino acid isoaspartyl-containing peptide centered around N15 that was used to immunize rabbits (see FIG. 3) is underlined, and homologous amino acids shared between this isoaspartyl-containing peptide and the peptide sequence surrounding N7 are starred. The methionine indicated is the first ATG common to all alternatively spliced HuD mRNAs, which differ at their 5′ ends. We have focused on the N-terminal region of HuD for the following reasons: 1) The full length HuD protein precipitates under isoaspartyl-forming conditions, making it very difficult to study. 2) The depicted region N-terminal to the RRM is believed to be unstructured and contains numerous potential isoaspartylation sites. However, we do not exclude that additional isoaspartylation sites might be present, for example in the region between RRM 2 and RRM3. B) (Left panel) Coomassie Blue staining used to verify presence of proteins after in vitro isoaspartyl conversion incubation and methylation reaction. Lanes 2 and 3 contain 1.1 μg and 0.34 μg of synapsin (˜80 kDa, considered to be a protein highly prone to isoaspartyl conversion), respectively. Lanes 4-11 contain approximately 0.75 μg of purified recombinant HuD (˜20 kDa, N-terminal fragment similar to A, above, containing RRM1 and upstream amino acids, but with a serine instead of glutamic acid at amino acid position 2 for cloning purposes and a hexahistidine tag at the C-terminus for purification purposes), incubated in isoaspartyl conversion buffer for 0-7 days. PIMT (˜25 kDa), added to detect isoaspartyl sites, was also used as loading control. (Right panel) Autoradiography of the gel. Isoaspartyl-converted synapsin and HuD were visualized by methylation with [3H]-AdoMet by PIMT. The HuD fragment is highly prone to conversion (compare with synapsin in lanes 2 and 3). This represents the autoradiogram after 46 hours of exposure. C) Determination that the isoaspartylation sites in the N-terminal region lie in the flexible portion and not in the RRM. Three proteins were incubated under isoaspartylation conditions for up to 7 days: the original 117 amino acid N-terminal fragment (with a serine at position 2 and a hexahistidine tag at the C-terminus), a similar fragment in which the 8 N and D residues in the 36-amino acid flexible region were mutated to Q and E respectively (residues that do not undergo isoaspartylation) and RRM1 lacking the N-terminal 36 amino acids. 1 ug of protein was loaded from Day 0 (control), day 3 and day 7 of the incubation and protein was visualized by Coomassie staining. A similar gel was blotted and subjected to on-blot methylation with [3H]-AdoMet by PIMT. Only the Day 3 and Day 7 samples from the original 117 amino acid fragment show above background labeling. HuD protein samples (1 μg) were resolved on NuPAGE® Novex 10% Bis-Tris Gel. A semi-dry transfer system was used for transfer to a PDVF membrane. The on-blot methylation reaction was performed with the following components: recombinant rat PIMT, 4 μM, [3H]AdoMet, 4 μM, 10,000 dpm/pmol, 0.1 mg/mL BSA, 30% methanol, 100 mM Na-MES, pH 6.2. The dry PVDF membrane after on-blot methylation was incubated with a storage phosphor screen for 24 h and radioactivity was detected by a Typhoon imager (GE Healthcare Life Sciences). In addition, those samples show the appearance of a higher molecular weight protein. This confirms that N and/or D residues in the N-terminal 36-amino acid region are prone to isoaspartyl conversion in vitro, but that N and D residues in the structured RRM1 do not convert. This makes sense because isoaspartylation, which kinks the protein backbone, cannot occur is the protein is highly structured. The labeling of the band at ˜30 kD in WT protein Day 3 and 7 (right panel), which can be detected despite the fact that there is very little of this complex (it is not visible protein on the coomassie gel), indicates that this higher molecular weight complex contains isoaspartylated residues. Some similar complex is seen in the last few lanes on the right in FIG. 2B.

FIG. 3 shows that the anti-isoaspartyl antibody specifically recognizes the isoaspartyl form of the HuD peptide. A) Schematic representation of antibody purification. B) ELISA analysis of pre-purified serum (a, 1:100,000 dilution), Column 1 flow-through (b, 1:100,000 dilution), Column 1 eluted antibody (c, 0.01 μg/ml), affinity-purified antibody (d, 0.01 μg/ml), and no antibody controls (left white and grey bars) against an unmodified and modified HuD peptide containing an isoaspartate residue at N15. The affinity-purified/absorbed antibody shows specificity for the isoaspartyl form of the peptide, as represented by the 3-fold increase in absorbance between the unmodified and modified forms of the peptide, while the absorbance remains similar when the cross reactive antibody is incubated against the two peptides. The ELISA data was provided by YenZym Antibodies, South San Francisco, Calif.

FIG. 4 shows that the isoaspartyl peptide-specific antibody specifically recognizes the isoaspartyl conversion of HuD in vitro. 2 μg of each HuD construct (amino acids 1-117 as in FIG. 2) was in vitro isoaspartyl converted at 37° C. for seven days, and time points were taken at day 0, 1, 3, and 7 and subjected to SDS-PAGE and Western blot analysis. Conversion reactions were set up in triplicate. Time course comparisons of in vitro isoaspartyl converted wildtype and mutants are displayed: wildtype versus single mutant N15Q and double mutant N7Q+N15Q. The mutant proteins have much weaker reactivity in comparison to the wildtype protein. The protein of expected size is ˜20 kDa (lower black bracket), and proteins of higher molecular weight appearing over the time course (upper gray bracket) may represent aggregated or di-/tri-merized protein that formed during the incubation. Coomassie Blue staining shows equal loading of the proteins throughout the time course (right panel). Bands tend to get diffuse over time, suggesting altered protein mobility. Experiments were performed in triplicate, and a representative example is shown here. Exposure time was five minutes.

FIG. 5 shows that HuD protein can become isoaspartylated in the absence of the repair enzyme PIMT in vivo. Brain lysate of PIMT knockout mice is examined because HuD is highly present in the brain. PIMT wildtype (^{+/+}) and deficient (^{-/-}) murine brain lysate analyzed for PIMT, HuD expression, and isoaspartylated HuD by Western blot. ˜30 ug of wildtype and deficient mouse lysate were used to verify the PIMT genotype using a commercially available antibody at a 1:250 dilution factor incubated overnight at 4° C.; exposure was performed by chemiluminescence using a suitable secondary IgG-HRP antibody for 3 minutes. This confirms that the knockout mice are indeed deficient for PIMT enzyme. In the right two panels, ˜60 ug of brain lysate was used to similarly analyze the presence of HuD and isoaspartylated HuD using a commercially available anti-HuD polyclonal antibody (1:250) with a complementary secondary IgG-HRP antibody (exposure 1 minute) and (in the rightmost panel) the rabbit antiserum raised against isoaspartylated HuD was used at a 1:400 dilution factor, a complementary secondary IgG-HRP antibody was used and the filter exposed for 14 seconds. The multiple bands at the HuD location in the middle panel could be splice isoforms or Hu family members that cross react with the anti-HuD antibody. Blots were stripped and reprobed with an anti-actin antibody (1:250, 1 minute exposure) to check for similar loading. Note the strong signal for isoaspartylated HuD in the brain of the PIMT knockout mice (rightmost lane), showing that these mice acquire isoaspartylated HuD because they cannot repair it. This demonstrates that HuD can become isoaspartylated in vivo.

FIG. 6 shows an exemplary immunohistochemical analysis of brain sections from PIMT wildtype (+/+) and knockout (−/−) mice. A) The anti-isoaspartyl Hu antiserum detects strong reactivity in PIMT^{-/-} mice where the PIMT enzyme is lacking, and shows weak reactivity in wildtype mice where the enzyme is strongly expressed. B) Peptide competition assay to determine the specificity of the affinity-purified/-absorbed isoaspartyl Hu antibody. The antibody was pre-incubated with 1000-fold molar excess of wildtype HuD peptide or an isoaspartyl-containing HuD peptide prior to performing immunohistochemistry. The peptide used was the same one used to generate the antiserum, see FIG. 2, underlined, and FIG. 3. The wildtype peptide does not compete out the antibody, whereas the isoaspartyl-containing peptide absorbs most of the antibody, indicating the antibody specificity for isoaspartylation. Positive staining is pink/fuschia. 20× magnification.

FIG. 7 shows an exemplary immunohistochemical analysis tumor sections from human SCLC patients (A) and anti-Hu antibody positive and negative SCLC-prone mice (B), and of brain sections from wildtype FVB/N mice (C). Hu staining is strong in both human and murine SCLC tumors (A and B), as well as in the brain of wildtype FVB/N mice (C). The isoaspartyl-specific antibody detects reactivity with tumor sections from human SCLC patients (A) and SCLC-prone mouse model (B). A) In the human SCLC samples, reactivity with anti-isoaspartyl antiserum is highest in the middle of the tumor, where cells are most likely deprived of nutrients. B) In the murine samples, reactivity with anti-isoaspartyl antiserum may be occurring in the same cells where Hu is expressed. PIMT expression is weak or negative in the three SCLC patients (A). PIMT staining is absent in murine SCLC tumors whereas Hu expression is high (B). C) The brain, where PIMT is normally highly expressed, shows strong signal for Hu and PIMT in the cerebral cortex and neural and glial cells (A). Positive staining is pink/fuschia. 20× magnification.

FIG. 8 shows exemplary PIMT expression in normal human lung, cortex, and hippocampus lysates using Western blot analysis. There is little to no Hu expression in the hippocampus, cortex, and lung lysates. The bands at different heights suggest that the antibody may be recognizing different splice isoforms or different members of the Hu protein family. PIMT is highly expressed in the neuronal samples. Actin was used to equalize loading. Hu protein is not expected to be expressed in normal lung, it is considered “abnormal” in the SCLC tumors. This shows that PIMT expression is low in normal lung.

FIG. 9 shows the result of an exemplary peptide competition assay to examine the specificity of the affinity-purified/-absorbed isoaspartyl Hu antibody. The antibody was pre-incubated with 0 or 1000-fold molar excess of wildtype HuD peptide or an isoaspartyl-containing HuD peptide (peptides were the same amino acid sequence as the peptide used to generate the antibody, see FIGS. 1 and 3) prior to performing Western blot analysis on in vitro isoaspartyl-converted HuD wildtype protein. Excess wildtype peptide does not compete out the antibody well, whereas excess isoaspartyl-containing peptide absorbs most of the antibody, indicating the antibody specificity for isoaspartylation. Exposure time shown here is 2.5 minutes.

FIG. 10 illustrates Hu protein family homology and cross-reactivity with anti-isoaspartyl Hu antiserum. A) Upper panel shows a comparison of N-terminal HuD amino acid sequence with that of other Hu protein family members and with the sequence of the peptide used to develop the rabbit anti-isoaspartyl HuD antiserum. Note that the cysteine at the N-terminus if the peptide was added for coupling purposes. The peptide sequence is lined up to areas showing homology. HuB shows the greatest similarity to HuD. Grey; conserved amino acid type, yellow: identical amino acid. Black highlight: beginning of RRM1. Green HuR has little overlap, and when lined up there is a D in the N position. B) shows western blot of HuB, HuC and HuR subjected to isoaspartylation conditions for 0, 1, 3 of 7 days. HuB most strongly cross reacts with the antiserum, while only the band of higher molecular weight of HuC shows some reactivity, indicating that perhaps the aggregated protein has accumulated isoaspartyl residues. HuR shows no reactivity. Exposure time shown here is five minutes. C) Shows the corresponding Coomassie staining of parallel gels

FIG. 11 Immunization of mice with native and isoaspartylated HuD₁₋₁₁₇. To examine the immunogenicity of isoaspartylated HuD in comparison to native HuD, recombinant HuD₁₋₁₁₇ (same fragment as used throughout the figures, e.g. FIG. 2C) was incubated at physiological conditions (pH 7.4, 37° C.) for 7 days to induce isoaspartyl conversion, and injected into mice to measure antibody and T-cell responses. ˜150 ug of protein in 100 ul was used to make an oil-in-water emulsion with 100 ul incomplete Freund's adjuvant and injected (˜200 uL) subcutaneously into mice (n=5). Boosting was performed 7 days later with a similar amount of protein in a similar manner. Blood samples were taken at days: 0, 6, and 17 to examine the antibody response. The spleen was harvested 10 days after boosting to analyze isoAsp-HuD T-cell responses. Native HuD challenge was similarly tested so that we could compare the anti-HuD immune response; PBS immunization was used as a negative control. A) Comparison of anti-HuD antibody response by Western blot analysis between mice immunized with native HuD (top) and isoaspartylated HuD (bottom). Using isoaspartylated HuD triggered an anti-HuD antibody response even without the boosting injection, while under those conditions no anti-HuD antibody was detected in mice (n=5) immunized with native HuD; boosting was required to trigger an anti-HuD antibody response when native HuD was used for immunization (one mouse from each group shown for comparison). Mouse plasma was tested by Western blot at a 1:2000 dilution factor against ˜1 ug of native and isoaspartylated HuD. Exposure time was less than a minute by chemiluminescence. B) Levels of anti-HuD T-cell responses using a cellular T-cell proliferation assay (³H-incorporation). Mice (n=5) immunized with isoaspartylated HuD showed in vitro stimulation against isoaspartylated and native HuD; mice immunized with native HuD showed weak reactivity similar to mice immunized with PBS. Two mice from each group are shown for comparison purposes.

FIG. 12 A collection of human SCLC serum (n=35) was used to narrow the HuD reactive epitope. HuD recombinant fragments (1-117 aa) and (37-117 aa) were used in Western blots to detect reactivity of SCLC patient serum. ˜1 ug of each recombinant protein that had been incubated for 0, 3, and 7 days at physiological conditions (pH 7.4, 37° C.) were resolved by SDS-gel electrophoresis and transferred to a PVDF membrane, human serum was used at a 1:1000 dilution factor and incubated over night at 4° C., a secondary anti-human IgG-HRP antibody was used at a 1:2000 dilution factor. Blots were exposed using a chemiluminescence reagent for 5 minutes. Three representative blots are shown. These analyses indicate that the first 36 amino acids of HuD (depicted in FIG. 2A), the region we have identified as isoAsp-prone region, are a target for human SCLC serum reactivity.

### DETAILED DESCRIPTION

### Definitions and Abbreviations

AdoMet=S-adenosyl-L-methionine

PEM/SN=paraneoplastic encephalomyelitis/sensory neuronopathy

PIMT=protein-L-isoaspartyl (D-aspartate) O-methyltransferase

PNS=paraneoplastic syndrome

RRM=RNA recognition motif

SCLC=small-cell lung cancer.

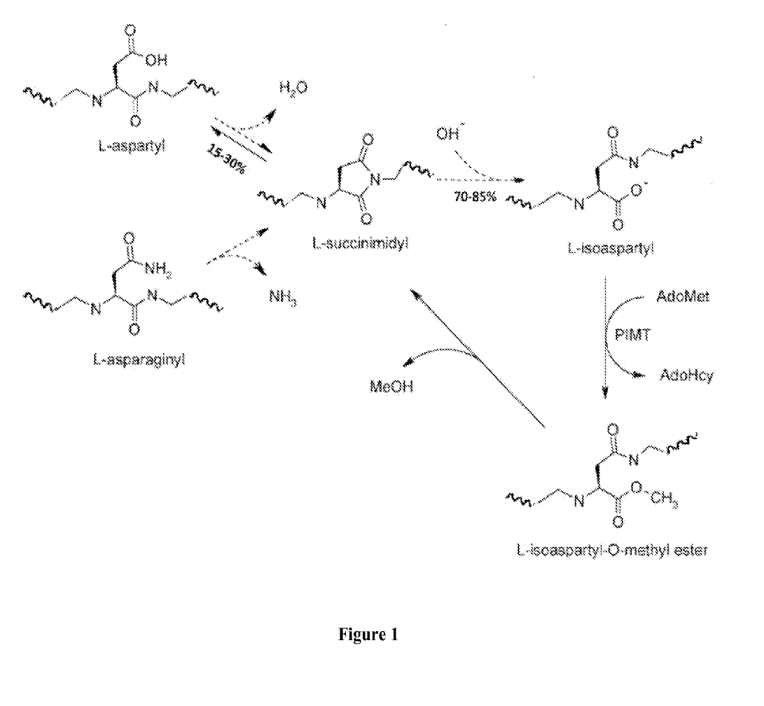

As used herein, the term “isoaspartylated proteins” refer to proteins that contain isoaspartate residues. Proteins containing asparagine (N) or aspartate (D) residues may undergo isoaspartylation in physiological conditions as illustrated in FIG. 1. Such proteins have been found in the present invention to possess antigenic properties, and to be present in the tumors of SCLC patients.

As used herein, the term “antigenic peptide fragment” refers to peptide fragments of isoaspartylated proteins which encompass at least one of the isoaspartate residues of the parent proteins. Such peptide fragments may retain the antigenic properties of the parent proteins and are, therefore, referred to herein as antigenic peptide fragments. Antigenic peptide fragments are generally in between 5 and 40 residues long, preferably about 10 to 20 residues long, more preferably about 15 residues long.

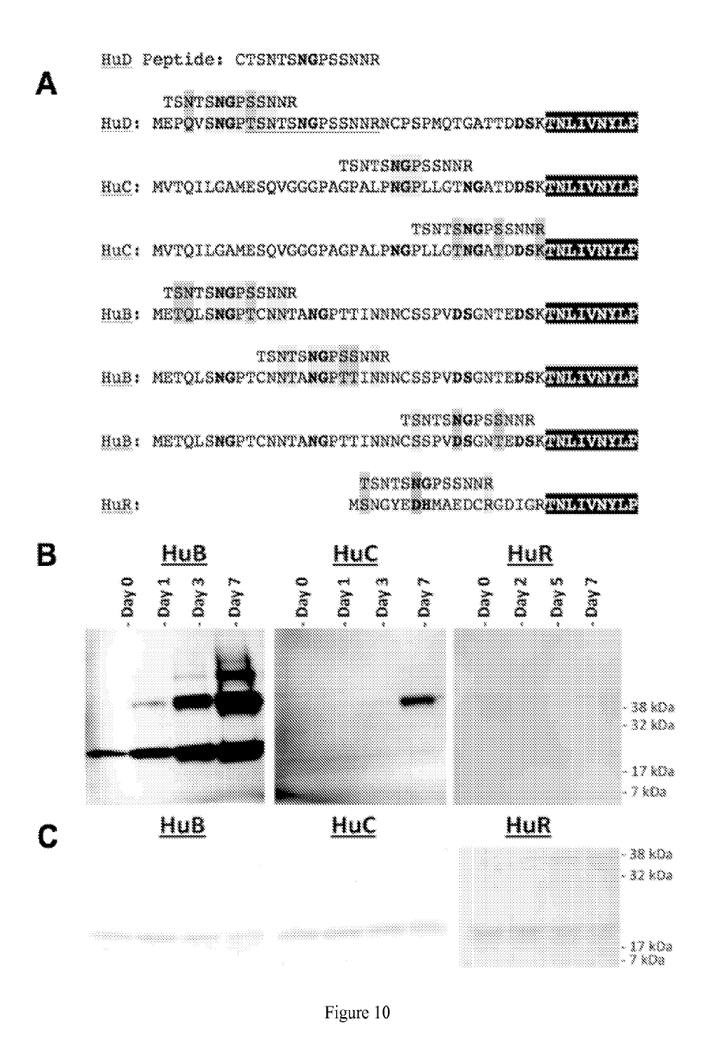

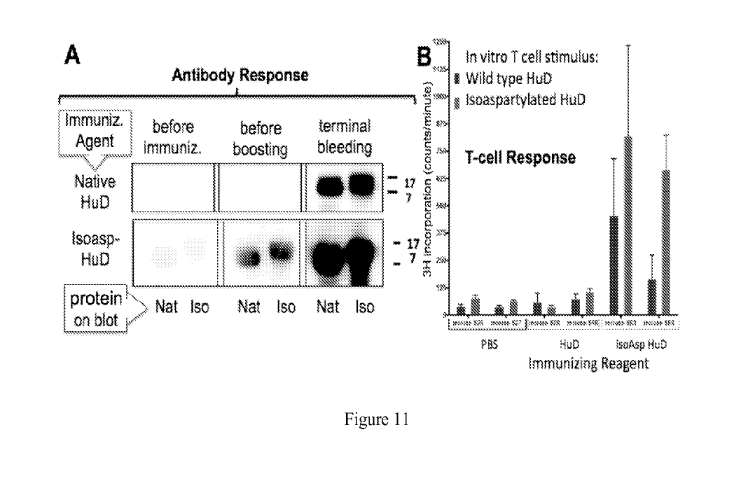

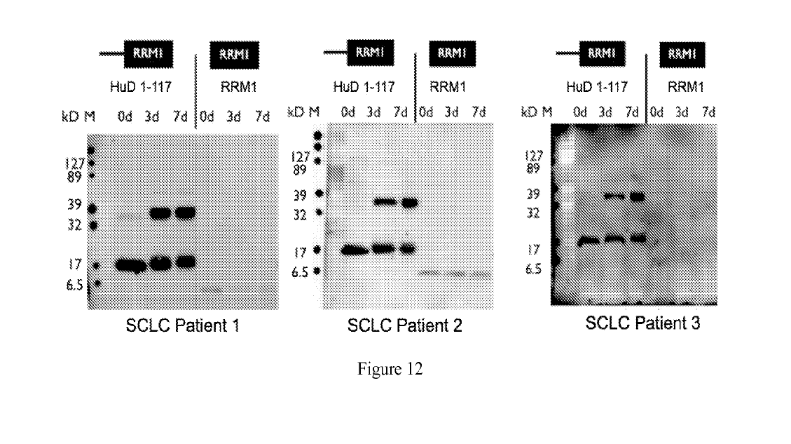

As used herein, the term “Hu protein” refers generally to any one of HuB, HuC, or HuD. A fourth member of the Hu protein family, HuR, is ubiquitously expressed and is not considered a SCLC antigen. (see Good P J. A conserved family of elav-like genes in vetrebrates, Proc Natl. Acad Sci USA 92: 4557-61, the entire content of which is incorporated herein by reference). Referring to FIG. 10 upper panel, there is shown a sequence alignment of the different members of the Hu protein family. The different variants of the Hu proteins all have an N-terminal region that appear to be antigenic. The N-terminal regions of HuB, HuC and HuC are defined by all residues that lie upstream of the first RRM domain. Splice isoforms of these protein exists that may differ in the exact composition and length of the N-terminal region, but they all contain N and D-containing sequences that lie adjacent to RRM1.

The term “Hu antigenic peptide” refers to a peptide of any length wherein the peptide has a sequence homology to a Hu protein. The homology between the peptide and the Hu protein is preferably between 60 to 80%, more preferably between 80 to 100% homologous to any Hu protein, so long as the key isoaspartate residues are preserved. We have shown that antigenic peptides can be generated from the N-terminal region of HuD, but other flexible regions of HuB, C and D that contain D or N residues, such as the hinge between RRM 2 and 3 are expected to have the potential provide antigenic peptides as well.

With respect to the isoaspartylated proteins or peptides, the present invention further embraces variants and equivalents which are substantially homologous to the parent proteins and peptides set forth herein. These can contain, for example, conservative substitution mutations, i.e. the substitution of one or more amino acids by similar amino acids. For example, conservative substitution refers to the substitution of an amino acid with another within the same general class such as, for example, one acidic amino acid with another acidic amino acid, one basic amino acid with another basic amino acid or one neutral amino acid by another neutral amino acid. What is intended by a conservative amino acid substitution is well known in the art.

In the context of the present invention, proteins, peptides, and nucleic acids may contain homologous or conservative substitutions and still remain “substantially the same.” The term “substantially the same” refers to nucleic acid or amino acid sequences having sequence variation that do not materially affect the nature of the protein (i.e. the structure, stability characteristics, substrate specificity and/or biological activity of the protein). With particular reference to nucleic acid sequences, the term “substantially the same” is intended to refer to the coding region and to conserved sequences governing expression, and refers primarily to degenerate codons encoding the same amino acid, or alternate codons encoding conservative substitute amino acids in the encoded polypeptide. With reference to amino acid sequences, the term “substantially the same” refers generally to conservative substitutions and/or variations in regions of the polypeptide not involved in determination of structure or function.

An “isolated” or “purified” polypeptide or protein is a polypeptide or protein that is found in a condition other than its native environment, such as apart from blood and animal tissue. In a preferred form, the isolated polypeptide is substantially free of other polypeptides, particularly other polypeptides of animal origin. It is preferred to provide the polypeptides in a highly purified form, i.e. greater than 95% pure, more preferably greater than 99% pure. When used in this context, the term “isolated” does not exclude the presence of the same polypeptide in alternative physical forms, such as dimers or alternatively glycosylated or derivatized forms.

As used herein, the phrases “treating cancer” and “treatment of cancer” mean to inhibit the replication of cancer cells, inhibit the spread of cancer, decrease tumor size, lessen or reduce the number of cancerous cells in the body, or ameliorate or alleviate the symptoms of the disease caused by the cancer. The treatment is considered therapeutic if there is a decrease in mortality and/or morbidity, or a decrease in disease burden manifest by reduced numbers of malignant cells in the body.

An “antibody” is an immunoglobulin molecule that recognizes and specifically binds to a target, such as a protein, polypeptide, peptide, carbohydrate, polynucleotide, lipid, etc., through at least one antigen recognition site within the variable region of the immunoglobulin molecule. As used herein, the term is used in the broadest sense and encompasses intact polyclonal antibodies, intact monoclonal antibodies, antibody fragments (such as Fab, Fab′, F(ab′).sub.2, and Fv fragments), single chain Fv (scFv) mutants, multispecific antibodies such as bispecific antibodies generated from at least two intact antibodies, fusion proteins comprising an antibody portion, and any other modified immunoglobulin molecule comprising an antigen recognition site so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

As used herein, the term “antibody fragments” refers to a portion of an intact antibody. Examples of antibody fragments include, but are not limited to, linear antibodies; single-chain antibody molecules; Fc or Fc′ peptides, Fab and Fab fragments, and multispecific antibodies formed from antibody fragments.

As used herein, “humanized” forms of non-human (e.g., murine) antibodies are chimeric antibodies that contain minimal sequence, or no sequence, derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies can comprise residues that are not found in the recipient antibody or in the donor antibody. These modifications are generally made to further refine antibody performance. In general, the humanized antibody will comprise substantially all of at least one, and typically two, variable domains, in which all or substantially all of the hypervariable loops correspond to those of a nonhuman immunoglobulin and all or substantially all of the FR residues are those of a human immunoglobulin sequence. The humanized antibody can also comprise at least a portion of an immunoglobulin constant region (Fc), typically that of a human immunoglobulin. Examples of methods used to generate humanized antibodies are described in U.S. Pat. No. 5,225,539 to Winter et al. (herein incorporated by reference).

The terms “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabelled A) in a reaction containing labeled “A” and the antibody will reduce the amount of labeled A bound to the antibody.

“Treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

As used herein, an “effective amount” is an amount sufficient to carry out a specifically stated purpose. An “effective amount” may be determined empirically and in a routine manners in relation to the stated purpose.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

Unless otherwise defined, all technical and scientific terms used herein have the same meaning as commonly understood by one of ordinary skill in the art to which this invention pertains. In case of conflict, the present document, including definitions, will control. All publications, patent applications, patents and other references mentioned herein are incorporated by reference in their entirety.

Having generally described the various aspects of the present invention above, a detailed description of the exemplary methods and reagents in accordance with the various aspects are provided:

### Methods for Making Isoaspartyl Antigen Recognition Agents

As stated above, one aspect of the present invention is directed to a method of forming an isoaspartyl antigen recognition agent that is capable of specifically recognizing and binding to an isoaspartylated protein or an antigenic peptide fragment thereof. Such antigen recognition agents may be useful for a number of applications including but not limited to diagnostic imaging reagents, immunotherapeutic agent, or research tools.

Methods in accordance with this aspect of the invention will generally include the steps of selecting a target protein; isoaspartylating the protein or a peptide fragment thereof; and developing or identifying a molecule that is capable of specifically binding to the isoaspartylated protein or fragments thereof to serve as the antigen recognition agent.

Preferred target proteins are those that may naturally undergo isoaspartyl conversion under physiological conditions. Proteins and peptides containing asparagine (N) and/or aspartate (D) residues may undergo isoaspartylation under physiological conditions, as illustrated in FIG. 1. To serve as a target for developing antigenic recognition agents, target proteins or peptides may be isoaspartylated in vitro. For example, purified proteins may be isoaspartylated, also known as “aging”, by incubating K-HEPES (pH 7.4), EGTA, sodium azide, and glycerol for 7 days at 37° C. (37,38). Other methods of isoaspartylating proteins or peptides known in the art may also be advantageously employed, such as generating isoaspartylated peptides through synthesis.

Exemplary antigen recognition agents may be made from a number of different classes of molecules including, but not limited to, proteins, antibodies, RNA aptamers, or any other small molecules with specific binding affinities for the isoaspartylated protein or peptide.

Depending on the particular class of molecule used to make the antigen recognition agent, those skilled in the art will readily understand that suitable methods for generating or developing such a molecule may be used. For example, if RNA aptamers are to be used to make the antigen recognition molecule, the SELEX protocol (see Tuerk C & Gold L (1990). Science. 249:505-510, the entire content of which is incorporated herein by reference) may be used to develop a specific aptamer that will specifically bind to the isoaspartylated protein or peptide fragment.

In some cases, in vitro isoaspartylation of full size proteins may be difficult as they may be precipitated out of the solution. Fortunately, it has been found that peptide fragments containing at least one of the isoaspartate residues of the parent protein may retain the antigenic property and may be used in place of the full size protein. Such peptide fragments are preferably between 5-40 residues long, more preferably between 10-30 residues long, or between 10-20 residues long. More preferably, the peptide is about 13 or 15 residues long.

In one preferred embodiment, the target protein is a HuD protein and the antigen recognition agent is an antibody. The antibody may be developed by immunizing a suitable animal (e.g. horse, mouse, humanized mouse, or rabbit) with the isoaspartylated protein or peptide fragment, collecting the serum, and isolating the antibodies from the serum.

Alternatively, the spleen could be used to generate hybridomas to produce monoclonal antibodies. Methods for generating monoclonal antibodies are known in the art. For example, monoclonal antibodies may be generated from a humanized mouse or may be generated by phage display.

In one exemplary embodiment, a humanized antibody may be produced by following the general steps of immunizing a humanized mouse with isoaspartylated Hu protein or protein fragments or peptides; generating monoclonal antibodies from the spleen of the immunized animals to make hybridomas; and selecting from said hybridomas those that produce monoclonal antibodies and react specifically with the isoaspartylated Hu protein or an antigenic peptide fragment thereof.

### Isoaspartyl Antigen Recognition Agents

In another aspect, the present invention is directed to an antigen recognition agent capable of specifically recognition and binding to an isoaspartylated protein or an antigenic peptide fragment thereof.

Isoaspartylated proteins of the present invention are preferably selected from those neuronal proteins that are also ectopically expressed in cancer cells. Exemplary neuronal proteins may include, but are not limited to the Hu proteins (HuB, HuC, and HuD), Sox proteins (Sox1, Sox2, Sox21), ZIC proteins (ZIC2, ZIC4), Nova-1, voltage-gated calcium or potassium channels (VGCC, VGKC), GABA receptors, nicotinic acetylcholine receptors, neuron-specific enolase, recoverin, synaptotagmin, synaptophysin, and other such proteins known in the art.

While the present invention is exemplified by SCLC, any other cancer cells that ectopically expresses a neuronal protein is within the scope of the present invention. For example, neuroblastoma is known to also express Hu proteins, which is expected to result in accumulation of isoaspartylated Hu proteins. Therefore, isoaspartyl antigen recognition agents described herein may also be applicable.

Methods for making isoaspartyl antigen recognition agents are as described above. In accordance with this the above described method for forming an isoaspartyl antigen recognition agent, such agent may be formed from any molecules capable of specifically binding to an isoaspartylated protein or an antigenic peptide fragment thereof. Exemplary classes of molecules may include but are not limited to proteins, antibodies, RNA aptamers, or any other small molecules with specific binding affinities for the iso-aspartylated protein or antigenic peptide fragment.

In some preferred embodiments, the antigen recognition agent is an antibody. In one embodiment, the antibody is a polycolonal antibody specific for isoaspartylated Hu protein or its antigenic peptide fragment. In a more preferred embodiment, the antibodies are rabbit polyclonal antibodies raised against a Hu protein antigenic peptide fragment containing one or more iso-aspartate residue(s). In a still more preferred embodiment, the polycolonal antibodies are capable of specifically recognizing isoaspartylated HuD protein or an antigenic peptide fragment thereof, wherein the HuD peptide contains isoaspartate residue at position N15, N7 or both, and the antigenic peptide fragment encompasses at least one of these isoaspartate residue.

The antibodies are preferably affinity purified to minimize cross-reactions with unmodified protein or peptide.

In another embodiment, the antibodies are humanized antibodies. Methods for forming humanized or monoclonal antibodies are generally known in the art and are not repeated herein.

### Methods for Early Detection of SCLC Cancer

In another aspect, the present invention provides a method for detecting SCLC cells. Methods in accordance with this aspect of the invention will generally include the steps of contacting a sample cell, such as cancer cells inside the patient, with an antigen recognition agent that specifically recognizes an isoaspartylated protein or an antigenic peptide fragment thereof. Preferably, the antigen recognition agent is a polycolonal antibody as described above or an antibody fragment thereof, and the isoaspartylated protein is isoaspartylated HuD protein. In some embodiments, the antigen recognition agent may include a signaling element, such as a fluorescent label or a radio-isotope or a chemical group, protein motif, or molecule that can be used to amplify the signal by reacting with other detection reagents, but are not limited thereto.

### Isoaspartyl Protein Tracers (Imaging Reagents)

In another aspect, the present invention also provides an imaging reagent for imaging the distribution of isoaspartylated proteins and antigenic peptide fragments thereof. Imaging reagents in accordance with this aspect of the invention will generally include an antigen recognition element operably linked to a signaling element.

In some preferred embodiments, the antigen recognition element is an antibody that binds specifically to an isoaspartylated protein or an antigenic peptide fragment thereof.

The signaling element may be any commonly known labeling element that can be used to label an antibody. Exemplary signally element may include, but are not limited to a fluorescent label, a radioactive label, or any other molecular imagining labels known in the art. The signaling element may be directly linked to the antibody or indirectly linked via a linker or a non-covalent bond.

### Method for Imaging the Distribution of Isoaspartyl Proteins

In another aspect, the present invention provides a method for imaging the distribution of proteins containing isoaspartate residues. Methods in accordance with this aspect of the invention will generally include the steps of introducing an imagining reagent as described above into a test sample; allowing the antigen recognition agent to bind to a target antigen; and then detecting location of the imaging reagent

### Targeted Therapeutic Agents for Treating SCLC and Methods for Using the Same

In still another aspect, the present invention provides a therapeutic agent for treating SCLC. Therapeutic agents in accordance with this aspect of the invention will generally include an antigen recognition element specific for a isoaspartylated protein or an antigenic fragment thereof. Preferably, the isoaspartylated protein is iso-aspartylated HuD protein.

In some embodiments, the antigen recognition element may be optionally linked to a payload, wherein the payload may be a cytotoxic agent consisting of a plant or bacterial toxin, a chemotherapy drug or a radioactive molecule. In some preferred embodiment, the antigen recognition element is a polyclonal antibody that binds specifically to a isoaspartylated Hu protein or a Hu antigenic peptide, preferably an iso-aspartylated HuD protein or a Hu antigenic peptide thereof.

The cytotoxic agent may be any suitable cytotoxic agent known to be capable of being coupled to an antibody. Exemplary cytotoxic agents may include yttrium-90, iodine 131, or calicheamicin, maytansin and auristatin (Beck A. et al, “The next generation of antibody-drug conjugates comes of age” 2010, Discovery Medicine 10: 329-39, the entire content of which is incorporated herein by reference).

In another embodiment the antigen recognition element is coupled to a molecule that stimulates the patient's own immune response to attack the tumor, such as IL-2 (Hombach A A and Abken H. 2012. Antibody-IL2 fusion proteins for tumor targeting, Methods Mol Biol. 907:611-26, the entire content of which is incorporated herein by reference) or other immunostimulatory molecules.

### Immuotherapeutic Compositions and Methods for Using the Same

In yet another aspect, the present invention provides an immunotherapy treatment method for treating SCLC. Methods in accordance with this aspect of the invention will generally include the steps of administering an effective amount of an immune-therapeutic agent that contains isoaspartylation or aggregates of the protein that accumulate under isoaspartylation conditions, this agent would be administered as described above to a patient who has been determined as suffering from SCLC with the objective of triggering an immune response to the tumor.

It will be understood by those skilled in the art that this aspect of the invention distinguishes from the prior aspect in that the immunotherapeutic agent and method described herein are isoaspartylated antigens that will trigger an immune response in the host against the tumor, thereby, acting much as a vaccine. In contrast, targeted therapeutic agents described in the prior aspect are designed to act as guided delivery vehicles that seek out isoaspartylated protein markers in the host.

### Theoretical Discussion

While not intending to be bound by any particular theory, the inventors believe that the following theoretical discussion will aid the reader to have a complete and full understanding of the present invention.

### Certain Experimental Observations and Insights

As explained above, SCLC is one of the most aggressive types of cancer. Several autoimmune diseases are associated with this cancer. In these rare syndromes, SCLC patients exhibit an immune response directed against neuronal proteins abnormally expressed in their tumors (2). When the native neuronal proteins also become targets, the autoimmune disease develops. Though such autoimmune syndromes are very uncommon, the characteristic antibodies are also found in a substantial fraction of SCLC patients without autoimmune disease (reviewed in Kazarian and Laird-Offringa, Molecular Cancer (2010) 10:33, the entire content of which is incorporated herein by reference). Why certain SCLC patients become immune responsive to neuronal proteins remains unresolved. The autoimmune antibodies typical for SCLC-associated paraneoplastic syndromes (PNS) might provide valuable insights into cancer-specific antigens, and may yield clues to aid in SCLC detection and therapy (reviewed in Kazarian and Laird-Offringa, supra). These antibodies and the mechanism by which they arise are therefore of great interest.

One of the autoimmune diseases associated with SCLC is paraneoplastic encephalomyelitis/sensory neuronopathy (PEM/SN) (3,4). SCLC patients with PEM/SN harbor high titers of antibodies directed against the neuronal Hu protein family (5). Hu proteins are RNA-binding proteins, three of which—HuB, HuC, and HuD—are normally expressed in the nervous system and gonads (6). In SCLC, however, they are ectopically expressed in the tumor. In PEM/SN patients, the immune system identifies neuronal Hu proteins as foreign, generating anti-Hu autoantibodies. Whether the antibodies play a role in the pathogenesis of the autoimmune disease was in question. Some researchers had raised the possibility that these antibodies react with native Hu proteins in the healthy nervous system, leading to PEM/SN in patients with high titer antibodies (7). Regardless, the process by which SCLC patients become immunoresponsive to the Hu proteins is hitherto unknown. Based on the sequence and structure of the Hu proteins, we hypothesized that, in the context of SCLC, these proteins might undergo a highly immunogenic post-translational modification called isoaspartyl conversion, and that this modification might trigger an autoimmune response in a subset of SCLC patients.

Isoaspartyl conversion occurs naturally under physiological conditions, usually at asparagines and aspartate residues that lie in flexible, unstructured regions of a polypeptide chain (8-10) and that are normally, but not necessarily, followed by a non-bulky side chain (glycine, serine, or histidine) (11-13). Isoaspartylation is more common in stressed and aged cells (“fatigued cells”) (8,14). The reaction occurs spontaneously through hydrolysis of aspartyl and deamidation of asparaginyl residues, generating a cyclic succinimide (FIG. 1) which opens to generate either an atypical isopeptide bond or a normal peptide bond (favoring the former at ˜7:3). In the isoaspartyl form, the polypeptide chain becomes linked through the β-carbonyl group, introducing a kink into the polypeptide backbone that may disrupt normal protein folding and activity and may contribute to protein instability.

Isoaspartyl-carrying proteins are continually repaired by the eukaryotic enzyme protein-L-isoaspartyl (D-aspartate) O-methyltransferase (PIMT). PIMT has widespread tissue and species distribution, suggesting that repair of isoaspartylated proteins is an essential function in cells (15,16). In mammals, PIMT is mainly cytosolic, and its specific activity is highest in the brain and testes (14, 15, 17-19), the location where HuB, HuC, and HuD proteins are normally expressed. PIMT catalyzes the transfer of the active methyl group of S-adenosyl methionine (AdoMet) onto the α-carboxyl group of atypical L-isoaspartyl sites (20,21) (FIG. 1). The resulting methyl ester rapidly decomposes to succinimide, which hydrolyzes to form a mixture of isoaspartyl and aspartyl residues. Each cycle of methylation/demethylation/hydrolysis typically repairs ˜25% of the isoaspartyl peptide bond by converting it to a normal aspartyl peptide. Unrepaired peptides are continuously recycled, and the overall repair efficiency is 85% or greater (14,17).

If conditions arise in which isoaspartyl moieties are not repaired, this can create neo-self antigens. Previous studies have indicated that isoaspartylated peptides are highly immunogenic and may elicit autoimmunity. For example, strong B and T cell responses characteristic of the autoimmune disease systemic lupus erythematosus (SLE) were elicited in mice immunized with the isoaspartyl forms of cytochrome c peptide and small nuclear ribonucleoprotein particle (snRNP), two major SLE antigens (22). In another example, H2B, a common autoantigen target in drug-induced lupus, has been shown to accumulate isoaspartyl residues, be a target of PIMT in vivo, and contribute to the onset of autoantibody production (23). These studies demonstrate that the isoaspartyl form of a self-peptide can be highly immunogenic even when T and B cells are originally unresponsive to the native form of the peptide. Thus, isoaspartyl forms of autoantigens occurring in vivo could stimulate the formation of autoantibodies that recognize the normal isoforms of these proteins, thus leading to autoimmunity (22,24).

A role of isoaspartyl residues in autoimmunity is further bolstered by analysis of PIMT knockout (−/−) mice. These mice show a hyperproliferative T cell response and develop autoantibodies and a systemic autoimmune pathology (25,26). Furthermore, they accumulate isoaspartate residues, show growth retardation, and succumb to fatal seizures (27,28). H2B from PIMT^{-/-} mice contains approximately 80 times the isoaspartyl content of H2B from wildtype mice (23). In vivo accumulation of isoaspartyl residues in H2B may explain why this histone is found as the major antigen in autoimmune diseases, such as lupus erythematosus (29-31).

Extending the above observation, we hypothesized that isoaspartylation may be a general mechanism of self-antigenic response which may explain the high titers of antibodies directed against the neuronal Hu protein family in SCLC patients. That is to say, based on the sequence of the N-terminal region of Hu proteins which possess potential sites of isoaspartylation, these proteins might be prone to isoaspartyl conversion and that their expression outside of their natural cell type or in the context of a cancer might increase their risk of remaining unrepaired. This could render them immunogenic, and in extreme cases, the immune response may spread to native proteins in the nervous system, thereby leading to paraneoplastic disease.

### Demonstrative Experiments

### Experimental Findings

Armed with the above observations and insights, we first undertook experiments to discover whether HuD, the neuronal Hu protein most commonly expressed in SCLC (32), undergoes isoaspartyl conversion. We demonstrate in this invention that residues in the N-terminal region of the protein, the region that has been previously implicated in immuno-reactivity in SCLC (32-35), are prone to isoaspartyl conversion in vitro. We further discovered that brain extracts and sections from PIMT^{-/-} mice show increased reactivity with an anti-isoaspartyl Hu antiserum, suggesting that Hu proteins can acquire this modification in vivo. Additionally, we uncovered the presence of this modification in human and murine SCLC tumor sections, providing evidence that isoaspartylated Hu proteins are indeed present in SCLC. We also show that immunization of mice with N-terminal HuD fragment carrying the isoaspartyl-prone region causes a much stronger immune response when the protein has been incubated under isoaspartylation-inducing conditions. Lastly, we show that serum from human SCLC patients is highly reactive with the iosaspartylation-prone region of HuD, as well as with higher molecular weight aggregates that form when the protein undergoes isoaspartylation.

### Experimental Procedures

Protein Purification—

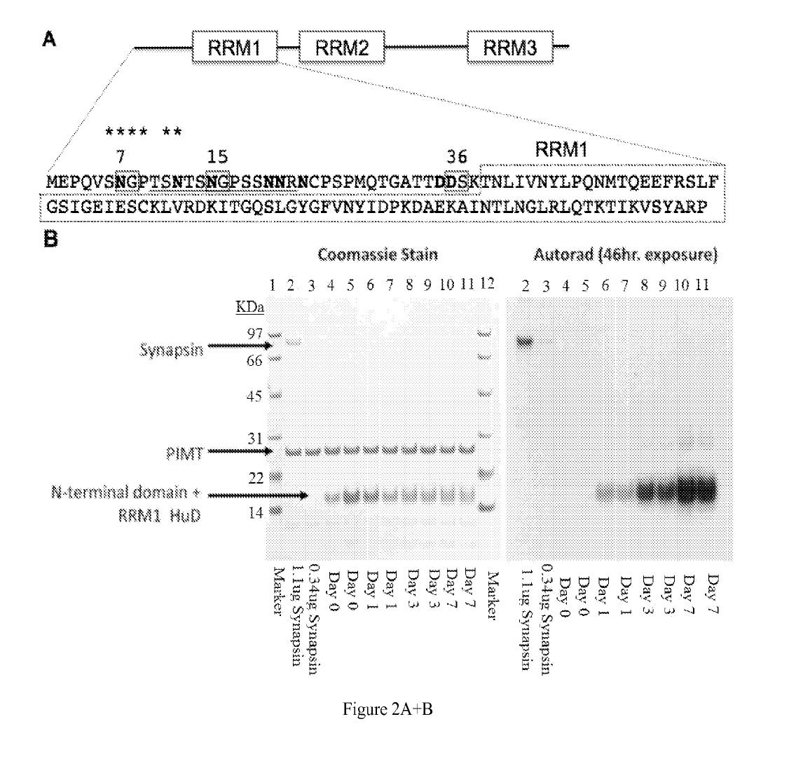

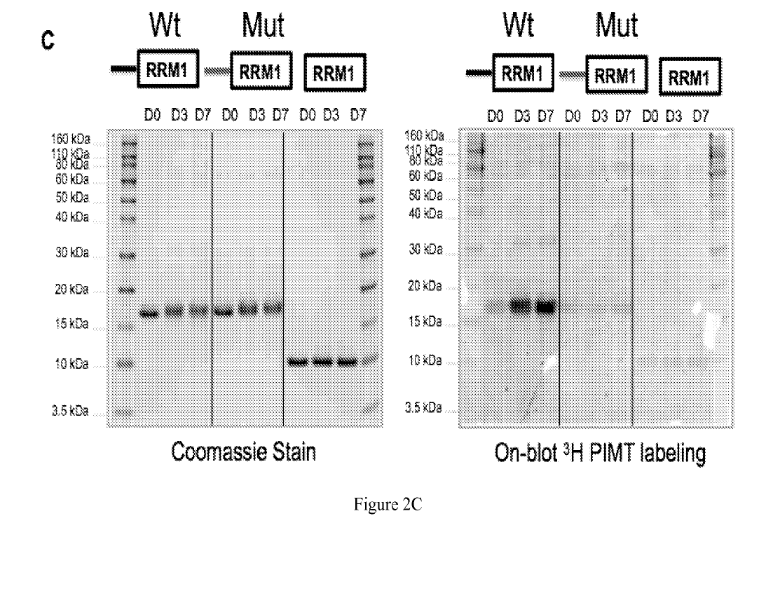

All recombinant Hu proteins were prepared as follows. Plasmids containing the N-terminal RRM1-containing regions of human HuB (amino acids (aa) 1-117), HuC (aa 1-117), HuD (aa 1-124), and HuR (aa 1-98) proteins were constructed as described in (36) and were transformed into a BL21 (DE3) variant containing a plasmid encoding two rare tRNAs (ArgU and ProL). Protein production was induced with 1 mM IPTG for four hours, and bacteria were pelleted and sonicated in buffer consisting of 20 mM HEPES (pH 7.4), 150 mM NaCl, and 0.5% Triton X-100. C-terminally His-tagged proteins were eluted from Ni2+ beads using sonication buffer containing 10% glycerol and 50-500 mM imidazole. Site-directed mutagenesis using PCR, restriction enzyme digestion, and custom oligonucleotide ligation was used to generate mutant constructs of the N-terminal domain of HuD containing the RRM1 encoding amino acids 1-124 (36). The canonical asparagines (N7 and N15) (for the purposes of this manuscript, canonical residues refer to asparagines or aspartate residues followed by glycine, serine, or histidine) were mutated to glutamines (Q7 and Q15). In FIG. 2C, all Ns and Ds in the N-terminal 36 amino acid region upstream of RRM1 were converted to Q or E respectively. Q and E do not become isoaspartylated. All constructs were verified by sequencing. Proteins were dialyzed at 4° C. in 50 mM potassium HEPES (pH 7.4) using the Slide-A-Lyzer Dialysis kit according to manufacturer's instructions (Thermo Scientific, Rockford, Ill.). After dialysis, the protein concentration was determined by the Bradford assay (Bio-Rad Laboratories, Hercules, Calif.) and verified on Coomassie blue-stained gels by running dilutions of the purified protein along a similar molecular weight standard of known concentration. All proteins were stored at −80° C.

In Vitro Isoaspartyl Conversion of Hu Proteins—

In vitro isoaspartyl conversion, also known as “aging”, was carried out by incubating purified protein (in a typical case one could use 2 μg in a final volume of 25 μl per time point) in 50 mM K-HEPES (pH 7.4), 1.0 mM EGTA, 0.02% (w/v) sodium azide, and 5% (w/v) glycerol for 7 days at 37° C. (37,38). Reactions were set up in triplicate, and time points were taken on days 0, 1, 3, and 7 and stored at −80° C. For the purposes of Western blot analysis and Coomassie-blue staining, 2 μg of protein (the entire volume taken for each time point) was loaded onto the gel. Reactions such as these can be set up with varying amounts of protein dependent on experimental needs.

Detection of In Vitro Isoaspartyl Conversion Using [3H]-S-adenosyl-L-methionine—

The repair pathway shown in FIG. 1 is the basis for in vitro assays of isoaspartate levels in proteins. Using [methyl-3H] AdoMet as the methyl donor, isoaspartate can be detected by [3H]-methyl incorporation into the protein by PIMT (38). This can be done by incubation in solution with PIMT followed by gel electrophoresis and imaging, as in FIG. 2B, or “on-blot” incubation with PIMT in which the reaction is carried out after gel electrophoresis and blotting, by incubating the blot with [methyl-3H] AdoMet and PIMT, as in FIG. 2C. For in solution isoaspartylation, in vitro isoaspartyl conversion reactions of 0.75 μg of the “aged” recombinant wildtype N-terminal domain of HuD containing RRM1 (amino acids 1-124) were subjected to a methylation reaction catalyzed by the recombinant rat protein-L-isoaspartyl methyltransferase (PIMT), using [3H]-S-adenosyl-L-methionine ([3H]-AdoMet) (Sigma-Aldrich, St. Louis, Mo.) as the methyl donor (see FIG. 1) (37,38). The incorporation of the radiolabeled methyl group was detected either by gel electrophoresis after the reaction (FIG. 2B) or by and blotting and on blot labeling with PIMT (FIG. 2C), followed by exposure to film (39,40). Radioactive signal indicates the presence of an isoaspartate residue that was repaired by PIMT (˜28 kDa). As a positive control, synapsin (˜80 kDa) (41,42) was also analyzed under the same conditions in FIG. 2B.

Anti-Isoaspartyl Hu Antiserum Generation—

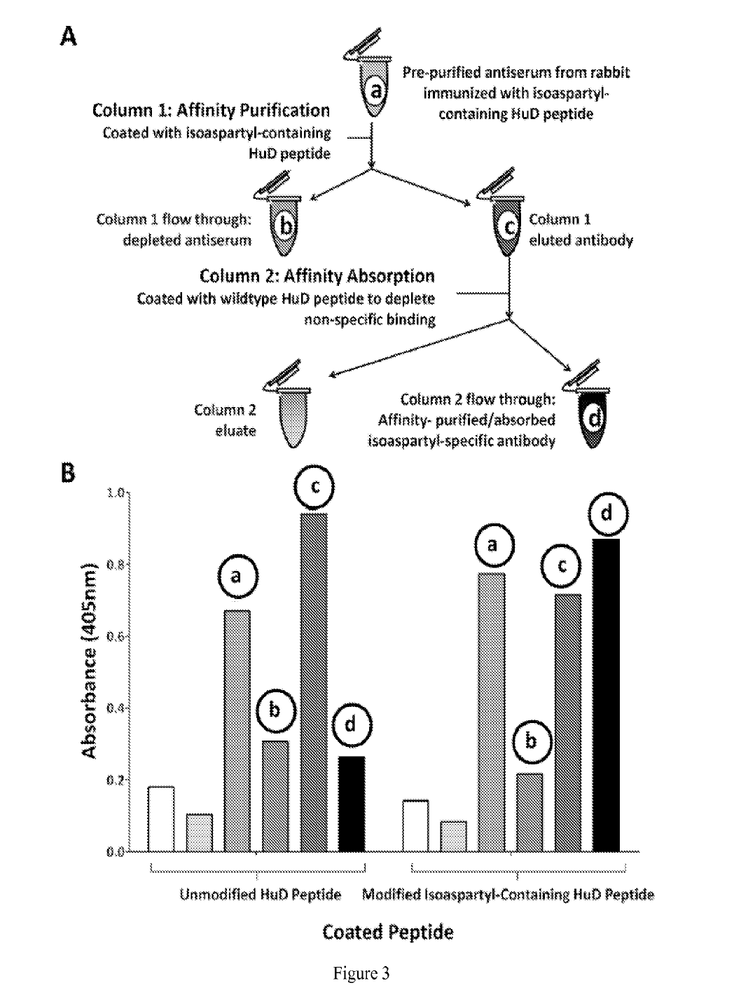

The sequence of the N-terminal domain of human HuD protein containing RRM1 was provided to YenZym Antibodies (South San Francisco, Calif.). The peptide containing the isoaspartyl form of N15 was selected as the antigen for rabbit immunization. Two rabbits were immunized with the following 13 residue peptide: CTSNTS-isoaspartyl-GPSSNNR-amide, conjugated to a carrier protein (via the added N-terminal cysteine) to render the epitope more immunogenic. The elicited antibody was then affinity-purified against the same modified isoaspartyl-containing peptide used for immunization. This step yielded an antibody preparation consisting mainly of the isoaspartyl peptide-specific antibody, plus a subpopulation of antibody targeting the shared epitope between the modified and unmodified peptides. Next, the affinity-purified antibody was affinity-absorbed against its unmodified peptide counterpart CTSNTSNGPSSNNR-amide to separate the isoaspartyl peptide-specific antibody from the cross reactive population. ELISA analysis was performed by YenZym Antibodies (South San Francisco, Calif.) to examine the specificity of the isoaspartyl peptide-specific antibody (see FIG. 3). Briefly, microtiter wells were coated with 0.1 μg/100 μl/well of peptide to capture antibodies. Serum dilutions of 1:1000, 1:10,000, and 1:100,000 were added. 1, 0.1, 0.01, or 0.001 μg/ml of cross reactive or affinity-purified antibody was added to the wells. Absorbance was measured at 405 nm using a plate reader.

Western Blotting—

For detection of in vitro isoaspartyl conversion with the anti-isoaspartyl Hu antiserum, proteins (2 μg/time point) were boiled for ten minutes, resolved on 14% sodium dodecyl sulfate (SDS) gels, and transferred to PVDF filters as described in Towbin et al. with the following modifications (43). Transfer was performed for 60 minutes at 100 V while chilling buffer with a cold pack. The filters were blocked with 5% milk in Tris-buffered saline, Tween-20 (TBST: 10 mM Tris/HCl pH 8.0, 150 mM NaCl, 0.05% Tween-20) for at least one hour at room temperature. Blots were incubated for one hour with 1 μg/ml of affinity-purified rabbit anti-isoaspartyl Hu antibody (YenZym, South San Francisco, Calif.) in 5% milk/TBST with gentle agitation. Blots were washed three times for ten minutes with TBST at room temperature. Secondary antibody, goat anti-rabbit IgG HRP conjugate (Santa Cruz Biotechnology, Santa Cruz, Calif.) was diluted 1:20,000 in TBST and added for one hour. After washing three times for ten minutes in TBST, the blots were submerged for three minutes in Millipore Immobilon Western Chemiluminescent HRP Substrate prepared according to the manufacturer's instructions (Millipore, Billerica, Mass.). The blots were imaged using Bio-Rad Fluor-S™ MultiImager, capturing images every ten seconds during a five-minute exposure time or the Fuji LAS-1000 Plus Gel Documentation System for exposure times noted in the figure legends.

For detection of isoaspartyl conversion in tissues, 15-20 μg of normal human cortex lysate (Protein Biotechnologies, Ramona, Calif.) and brain extract from PIMT^{+/+} and PIMT^{-/-} mice (provided by the Aswad laboratory) were subjected to Western blot analysis, as described above. For detection of PIMT expression, 15-20 μg normal human lung, cortex, and hippocampus lysate (Protein Biotechnologies, Ramona, Calif.) was subjected to Western blot analysis. Primary antibodies were added individually with the following conditions and allowed to incubate with gentle agitation: rabbit anti-isoaspartyl Hu (1:100) at 4° C. overnight; rabbit anti-human HuD (Zymed Laboratories, Inc.) (1:500) at room temperature for one hour; rabbit anti-histone H4 (Santa Cruz Biotechnology, Santa Cruz, Calif.) (1:200) at room temperature for one hour; rabbit anti-actin (1:500) at room temperature for one hour (Cytoskeleton Inc., Denver, Colo.); mouse anti-human PCMT1 (1:200) at room temperature for one hour (Santa Cruz Biotechnology, CA); rabbit anti-human Hu (Santa Cruz Biotechnology, CA) (1:200) at room temperature for one hour. Blots were incubated for one hour at room temperature with the appropriate secondary antibodies for one hour at room temperature with gentle agitation, either goat anti-rabbit IgG (1:20,000) or goat anti-mouse IgG (1:10,000). Restore Plus Western Blot Stripping Buffer was used to strip and reprobe blots according to manufacturer's instructions (Thermo Scientific, Rockford, Ill.).

Peptide Competition Assay—

Peptide competition was performed in order to confirm the specificity of the reactive band of the anti-isoaspartyl Hu antiserum. 1 ug/ml of antibody was pre-incubated with 20-5000-fold molar excess of a wildtype HuD peptide or an isoaspartyl-containing HuD peptide at 4° C. overnight with gentle agitation. Western blot analysis of in vitro isoaspartyl-converted HuD wildtype protein was performed using either the wildtype HuD peptide plus antibody or the isoaspartyl-containing HuD peptide plus antibody as described above. Immunohistochemistry incubations on brain sections from PIMT^{+/+} and PIMT^{-/-} were carried out with antibody that had been incubated with either of the two peptides as described in the next section.

Immunohistochemistry—

Brain sections from PIMT^{+/+} and PIMT^{-/-} mice (27) were provided by the Mamula laboratory. Brain and SCLC tumor tissue sections from SCLC-prone mice (Luc:p53F/F; Rb1F/F) (44) were supplied by the laboratory of Anton Berns at the Nederlands Kanker Instituut in Amsterdam, the Netherlands. Tissues were fixed in 4% formalin in phosphate buffered saline solution and embedded in paraffin. 4 μm sections of tissues from SCLC-prone and 6 μm sections from PIMT^{+/+} and PIMT^{-/-} mice were prepared and rehydrated before staining. De-identified archival remnants of paraffin-embedded human SCLC tumor samples were purchased from the National Disease Research Interchange (NDRI) and given to the USC Pathology Core for sectioning (3 mm thickness) and mounting on charged glass slides. Haematoxylin and eosin stainings were performed according to standard procedures.

All tissues were analyzed by experienced pathologists who verified the identity of the tissue and assessed immunohistochemistry stainings to determine proper antibody dilutions. Immunohistochemistry experiments were performed by the USC Department of Pathology Immunohistochemistry Core according to standard procedure. Antibody titration experiments were performed to determine the best antibody conditions with the most specificity and least amount of nonspecific cell staining. Sections were individually incubated with the following primary antibodies: a) rabbit anti-human Hu 1:100 (Santa Cruz Biotechnology, Santa Cruz, Calif.); b) rabbit anti-isoaspartyl Hu 1:500 (0.5 ug/ml final concentration; YenZym, South San Francisco, Calif.); c) rabbit anti-human PIMT 1:500 (brain) or 1:1000 (lung, SCLC) (supplied by the Mamula lab at Yale University). Pre-diluted secondary antibodies from the Chemicon IHC SelectÒ Immunoperoxidase Secondary Detection System (Millipore, Billerica, Mass.) were used, and Streptavidin-HRP from the Chemicon IHC SelectÒ Immunoperoxidase Secondary Detection System (Millipore, Billerica, Mass.) with Chromogen Reagent (prepared according to manufacturer's instructions), which causes fuchsia staining, was used for detection. Haematoxylin counter stain solution was applied. Images were captured using a Zeiss Imager.Z1 microscope (Carl Zeiss Microscopy, Hertfordshire, UK) equipped with a Zeiss AxioCam digital camera and processed using AxioVision 4.6.3 software (Carl Zeiss Vision, San Diego, Calif.). Stained sections were visualized at 20× magnification.

### Results

The SCLC Autoantigen HuD is Prone to Isoaspartyl Conversion In Vitro—

In vitro incubation of purified proteins and peptides at pH 7.4 and 37° C. is a common method to test proteins for their propensity to accumulate isoaspartyl sites under physiological conditions (45). Of the three neuronal Hu proteins, HuD is most commonly expressed in human SCLC tumors (32). Therefore, recombinant HuD protein was incubated at pH 7.4 and 37° C. for seven days. Initially, the experiment was performed with full-length HuD, which contains eight potential conventional isoaspartyl-prone sites (Asp or Asn followed by Gly, Ser, or His). However, this protein quickly precipitated under these experimental conditions and was not used for further experiments (data not shown). Because the N-terminal domain of HuD containing RNA recognition motif 1 (RRM1) has been implicated in immune response in SCLC patients (32-35), we proceeded with this segment of the protein (amino acids 1-124). It contains three potential canonical isoaspartyl conversion sites in a putatively unstructured region upstream of RRM1 (N7, N15, and D36) (FIG. 2A). Samples were removed during the incubation period on days 0, 1, 3, and 7 and tested for their ability to accept a radiolabeled methyl group (3H-AdoMet) in a PIMT-catalyzed reaction. As shown in the autoradiogram in FIG. 2B, HuD exhibited no reactivity at Day 0, followed by a strong increase in PIMT-mediated methylation over the seven day incubation. This indicates formation of isoaspartyl sites under conditions of physiological pH and temperature. Interestingly, HuD was much more prone to conversion than synapsin, which was used as a positive control; synapsin is a protein that has been previously described to undergo this modification (38,41). From these data, we conclude that the N-terminal domain of HuD is highly prone to isoaspartyl conversion in vitro. To determine whether this process also occurs in vivo, we raised a rabbit anti-isoaspartyl Hu antiserum.

Generation of an Anti-Isoaspartyl Hu-Specific Antiserum—

The canonical isoaspartyl-prone residue N15 was deemed to be the best choice for peptide generation based on its predicted antigenicity, hydrophilicity, secondary structure, and potential for post-translational events. Rabbits were immunized with a peptide containing an isoaspartyl residue at N15: CTSNTS-isoaspartyl-GPSSNNR (SEQ ID. No 1) (FIG. 2A, underlined). Serum was collected and affinity purified on a column carrying the isoaspartyl-containing peptide, followed by affinity absorption of non-isoaspartyl-specific reactivity on a wildtype HuD peptide column (FIG. 3A). Whereas the antibody purified from the first column recognized both the wildtype and isoaspartyl form of the peptide (FIG. 3B, c), following immuno-absorption, the antibody showed considerable specificity for the isoaspartyl-containing HuD peptide as demonstrated by ELISA (FIG. 3B, d).

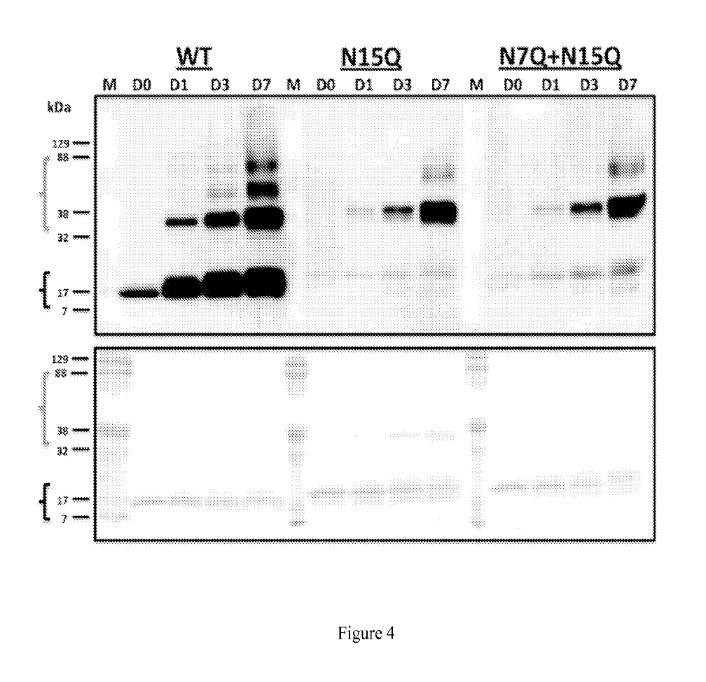

To verify the ability of the affinity-purified/-absorbed antibody to specifically recognize isoaspartyl HuD, we examined its reactivity against the N-terminal HuD fragment, isoaspartyl-converted as in FIG. 2B. As a negative control, we compared reactivity of the wildtype protein against a single N15 mutant and an N7 and N15 double mutant; these potential isoaspartyl sites were mutated to glutamines, the amino acid similar in property to the wildtype protein but not prone to isoaspartyl conversion. The peptide used to generate the antiserum contained an isoaspartate at the N15 position. The protein sequence in this region is partially homologous to the region containing N7 (see FIG. 2A, starred). Following incubation under in vitro isoaspartyl conversion conditions, the wildtype and mutant proteins were subjected to SDS-PAGE and Western blot analysis using the anti-isoaspartyl Hu antiserum (FIG. 4). There was some reactivity at day 0 for the wildtype protein, possibly due to residual cross reactivity of the antibody with the unconverted peptides. However, over the time course, there was a dramatic increase in reactivity in the HuD N-terminal fragment. In the mutant proteins, reactivity was very weak compared to the wildtype protein, suggesting that the antiserum was quite specific for the N15 site. However, some reactivity still accumulated over time (FIG. 4), possibly the result of conversion of the D36 site, the NG site within the RRM1 domain, and/or other non-canonical isoaspartyl conversion sites in the region N-terminal to RRM1 (see FIG. 2A). Additionally, reactivity against bands of higher molecular weight, presumably consisting of aggregated protein (46), became apparent during the time course (FIG. 4).

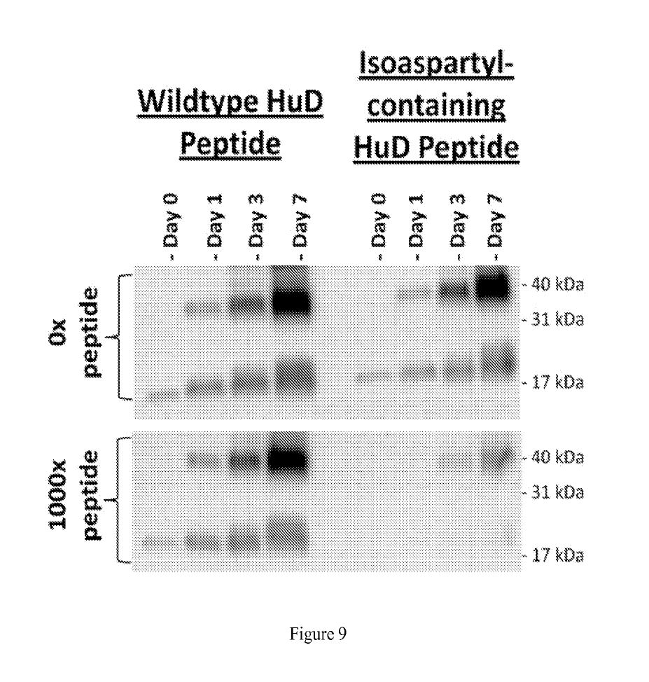

To further examine the isoaspartyl specificity of the antibody, we incubated the antiserum with either a 1000-fold molar excess of wildtype or isoaspartyl-containing HuD peptide (FIG. 9). The latter almost completely competed out recognition of in vitro isoaspartyl-converted wildtype HuD protein, indicating that the antiserum shows substantial specificity for the post-translational modification. Next, we examined the reactivity against the N-terminal RRM1-containing domain of HuB, HuC, and HuR (FIG. 10). The antiserum was highly reactive against HuB, which carries the amino acid sequence CNNTANGPTTINN (SEQ ID No. 2), in which five amino acids are identical (bold) and two conserved (italics) to the HuD segment against which the anti-isoaspartyl Hu antiserum was raised. Reactivity against HuC, which is less similar to HuD, was much weaker, only showing a higher molecular weight band. This suggests that though crossreactivity of the antiserum with HuC is weak, aggregated protein with isoaspartyl sites is present and at least somewhat cross-reactive. HuR showed no reactivity. Taken together, we conclude that at least HuB and HuD are highly prone to isoaspartyl conversion in vitro and that our antiserum recognizes the isoaspartyl form of both HuB and HuD proteins.

Anti-Isoaspartyl Hu Antiserum is Able to Detect Isoaspartyl Conversion In Vivo—

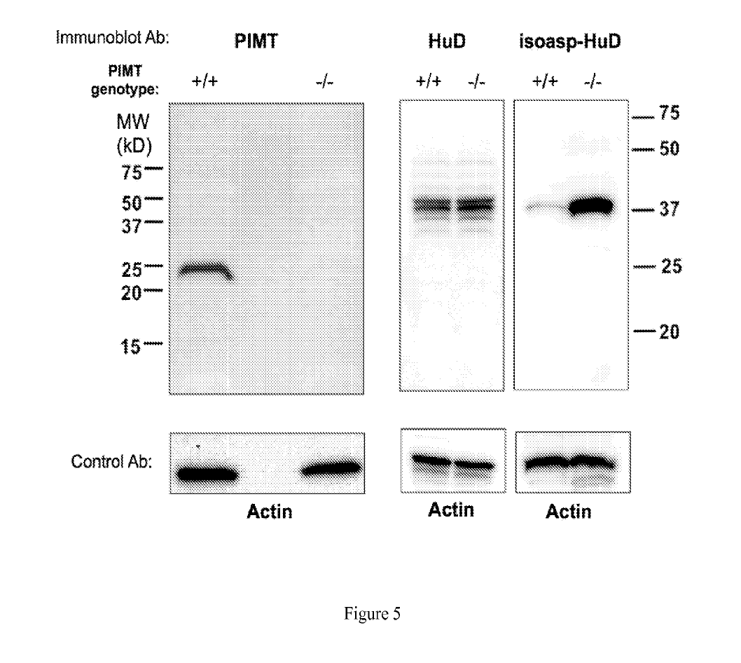

In order to examine whether isoaspartyl conversion occurs in vivo, first we used Western blot analysis to examine reactivity of the anti-isoaspartyl Hu antiserum with brain lysates from wildtype mice (PIMT^{+/+}) and mice in which PIMT had been knocked out (PIMT^{-/-}) (27) (FIG. 5). Isoaspartyl-containing proteins have been shown to accumulate to high levels in the brain tissues of PIMT^{-/-} mice (28,47). When comparing brain extracts for the two genotypes of mice, both showed similar levels of reactivity with the anti-Hu antibody (additional bands likely represent protein from alternatively processed HuD proteins or cross reactivity with the highly conserved neuronal Hu protein family members HuB and HuC). However, reactivity with the isoaspartyl-specific serum was much more apparent in the lysates from the PIMT^{-/-} mice. A prominent band at the size of Hu protein was visible in the blot probed with the isoaspartyl-specific antiserum. This suggests that Hu proteins had undergone isoaspartyl conversion in these mice and had not been repaired due to the lack of PIMT.

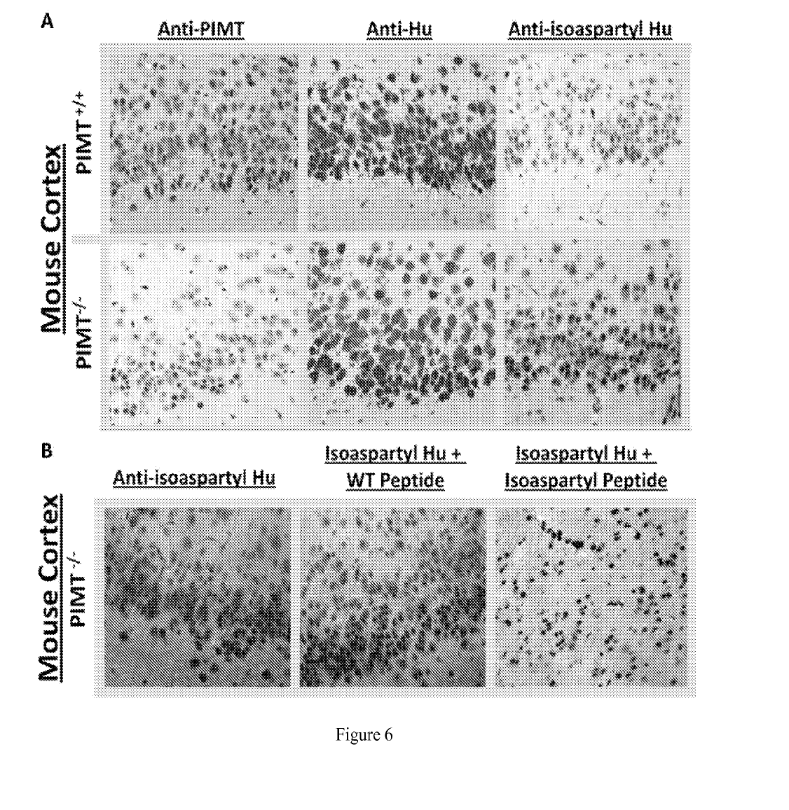

Next, we performed immuno-histochemistry to examine PIMT, Hu, and isoaspartyl-Hu expression in brain sections from PIMT^{+/+} and PIMT^{-/-} mice. As expected, PIMT staining was strong in the cortex of PIMT^{+/+} mice, with PIMT^{-/-} mice showing weak staining (these mice are confirmed to be genotypically PIMT negative). Neuronal Hu was strongly expressed in the brains of both PIMT^{+/+} and PIMT^{-/-} mice (FIG. 6A), and there appeared to be little difference in the level of expression when comparing the two genotypes. Reactivity with anti-isoaspartyl Hu antiserum was higher in the PIMT^{-/-} mice, though there was some weak staining in the PIMT^{+/+} mice (FIG. 6A). In a peptide competition assay, where the anti-isoaspartyl Hu antiserum was pre-incubated with either a wildtype or isoaspartyl-containing HuD peptide, the isoaspartyl peptide strongly competed out the anti-isoaspartyl Hu antiserum, whereas in the presence of the wildtype peptide, the signal was still detectable, indicating that the antiserum is highly specific for the modification (FIG. 6B). From the Western blot and immunohistochemistry results, it is evident that the isoaspartyl-Hu antiserum shows increased reactivity with tissues from PIMT deficient mice, strongly suggesting that the Hu protein undergoes isoaspartyl conversion in vivo.

Anti-Isoaspartyl Hu Antiserum Shows Reactivity with SCLC Tumor Sections—

We next asked whether isoaspartyl-converted Hu might be present in SCLC tissues. Based on the anti-Hu immune response seen in 15-25% of SCLC patients (48-52) and in a similar fraction of SCLC-carrying mice (35), we hypothesized that isoaspartyl-Hu would be present in at least a fraction of SCLC tumors and that it might not be properly repaired due to a relative lack of PIMT or lack of access of the enzyme to the kinked Hu protein, for example in areas of necrosis.

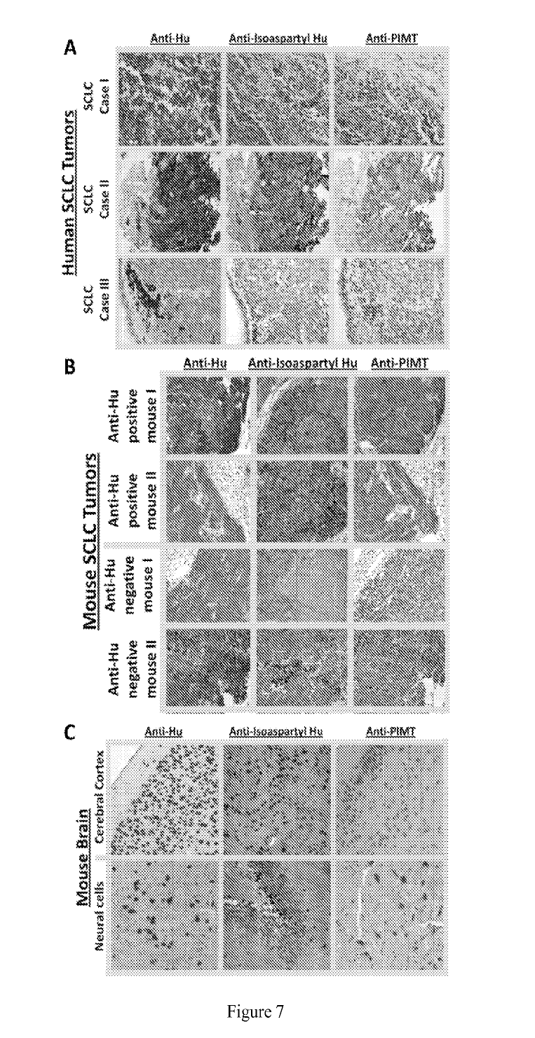

Next, we examined SCLC tumors from human SCLC patients and SCLC-prone mice (44,53) by immunohistochemistry (FIG. 7). Murine tumors were obtained from mice in which SCLC was induced by conditional knockout of floxed Rb and Trp53 genes through intratracheal instillation of Adeno-Cre virus (44,53). First, we confirmed the presence of Hu proteins in the tumors. Anti-Hu reactivity was high in SCLC tumors from both humans and mice (FIGS. 7A and B), in agreement with the observation that all SCLC tumors express neuronal Hu antigens (5,54). Probing with anti-isoaspartyl Hu antiserum showed variable reactivity in the tumors (FIGS. 7A and B). In general, this reactivity was weak and less uniform than that of the Hu staining. Reactivity with anti-isoaspartyl antiserum appeared to be more prominent in areas of necrosis or in the middle of the tumor where cells are prone to be deprived of oxygen and/or nutrients, supporting the notion that this modification occurs highly in stressed cells (8,14).

We had previously reported that, just as with human SCLC patients, a fraction of mice with SCLC showed anti-Hu immune reactivity (35). We hypothesized that isoaspartyl conversion of Hu proteins might trigger this SCLC-related anti-Hu immuno-responsiveness. Therefore, we compared the differences in anti-isoaspartyl Hu staining between two anti-Hu antibody positive and two anti-Hu antibody negative SCLC-prone mice (35). Reactivity with anti-isoaspartyl antiserum appeared to be more prominent in SCLC tumors from the anti-Hu antibody positive mice than in those from the anti-Hu antibody negative mice, which seemed to show weaker overall staining restricted to areas of necrosis (FIG. 7B). While this hints at a possible connection between levels of isoaspartyl-Hu and anti-Hu immune reactivity, a much larger number of mice would need to be studied to establish a conclusive link between the two parameters. Taken together, these data suggest that Hu proteins may be prone to isoaspartyl conversion in vivo in SCLC tumors.

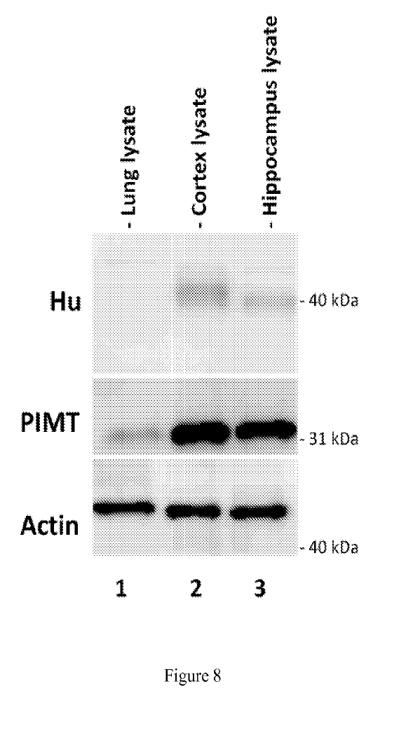

PIMT is expressed throughout the body, but is highest in the brain (14, 15, 17-19). We wondered what PIMT expression levels would be in SCLC, and whether there might be a correlation between the level of expression of PIMT and the presence of isoaspartyl-Hu in SCLC tumors. Examination of PIMT expression in tumor sections showed little staining in human SCLC tumors (FIG. 7A). Hu proteins were expressed in all three patients, but PIMT and isoaspartyl-Hu reactivity was absent (Case I) or weak (Case II and III). Likewise, PIMT staining was weak or absent in SCLC tumors from both anti-Hu antibody positive and negative SCLC-prone mice (FIG. 7B). In contrast, the PIMT antibody showed strong staining in the cortex and neural cells of these mice (FIG. 7C) and in the brains of healthy humans (data not shown). We also examined PIMT expression by Western blot (FIG. 8), which revealed that the expression level was high in the cortex and hippocampus, where PIMT activity is normally greatest (14, 15, 17-19), and low in the lung.

HuD is a neuronal protein that becomes misexpressed in all SCLC tumors (32). A small fraction of SCLC patients develop autoantibodies against this protein (48-52). What triggers this response is currently unknown. We hypothesized that Hu proteins might be prone to isoaspartylation, an immunogenic post-translational modification (22-28) that may trigger the immune response observed in anti-Hu autoantibody positive SCLC patients. To our knowledge, this is the first investigation examining isoaspartyl conversion of a SCLC-associated autoantigen.

We provide evidence that the SCLC-associated autoantigen HuD is highly prone to isoaspartyl conversion in vitro (FIGS. 2 and 4), and that HuD is a substrate for the repair enzyme PIMT in vitro (FIG. 2B) and in vivo (FIGS. 5 and 6). Previous data from human and mouse SCLC antiserum suggest that the N-terminal domain RRM1 of the HuD protein may be the key target of the autoimmune response (32-34). The domain of HuD N-terminal of RRM1 contains three potential canonical isoaspartyl conversion sites. Within this region, there are also a number of non-canonical sites, including N20. This residue is of interest because it is part of an asparagine-rich NNXN sequence, and recent studies reported that this sequence may favor asparagine deamidation (55,56). There is an additional potential isoaspartyl site within the RRM1 domain, but based on reports that isoaspartyl formation may be prevented by conformational constraints (8-10), we hypothesized that the site within the RRM1 would less likely be prone to conversion (57). Indeed, the experiment in FIG. 2C indicates that the RRM1 domain alone shows no signal above background when incubated under isoaspartylation conditions and then subjected to PIMT modification.

We developed an anti-isoaspartyl antiserum by immunizing rabbits with the peptide sequence surrounding N15 (FIGS. 2A and 3). Recently, an antibody was developed against two potential isoaspartyl conversion sites in the translation repressor eukaryotic initiation factor 4E-binding protein 2 (4E-BP2), and it was shown to be highly specific for the modification (55). In our studies, ELISA (FIG. 3B) and competition analysis (FIG. 9) suggest that the affinity-purified antiserum exhibited considerable specificity for the isoaspartyl form of the HuD peptide, although there appears to be some residual cross-reactivity with the unmodified form of the peptide (FIG. 3B). This finding is similar to what is observed with the anti-isoaspartyl 4E-BP2 antibody (55) and in mice immunized with the isoaspartyl forms of cytochrome c peptide and small nuclear ribonucleoprotein particle (snRNP) (22). In the latter study, an antibody response was unable to distinguish between wildtype and isoaspartyl form of the proteins. This may explain why we observe some reactivity by Western blot analysis against HuD protein at day 0 (FIG. 4). We too see cross-reactive antibodies (FIG. 3B, c), necessitating affinity purification and absorption to obtain a specifically reactive antibody preparation (FIG. 3B, d).

The reactivity of mutant proteins lacking N15 or N7 and N15 was greatly diminished, suggesting that the antiserum appears to be largely specific for the location matching the peptide used for immunization surrounding N15. The weak but detectable increase in reactivity over time of these mutant proteins with the anti-isoaspartyl Hu antiserum suggests that the D36 site or a non-canonical aspartate or asparagines might undergo isoaspartyl conversion.

The ability of Hu proteins to undergo isoaspartylation in vivo was examined by analyzing brain lysates and sections from PIMT^{+/+} and PIMT^{-/-} mice using the rabbit anti-isoaspartyl Hu antiserum. PIMT^{-/-} mice exhibit a very strong increase in the levels of isoaspartyl converted proteins due to lack of the repair enzyme (27,28). In our studies, we were able to detect strong reactivity with the anti-isoaspartyl Hu antiserum in PIMT^{-/-} brain lysates by Western blot analysis (FIG. 5) and in brain sections by immunohistochemistry (FIG. 6), supporting the idea that isoaspartyl conversion of Hu proteins occurs in vivo. As is observed in FIG. 6, a low level of isoaspartyl residues has been shown to be present in the brains of wildtype mice (27, 28, 58), indicating that not all damaged proteins are immediately repaired. Peptide competition on PIMT^{-/-} brain sections showed that isoaspartyl peptide strongly competed with the antibody, whereas the wildtype peptide did not compete as strongly (FIG. 6B), supporting the notion that the signal observed in the brain was specific for isoaspartyl conversion of Hu in vivo. Previous proteomic analysis using two-dimensional (2-D) gel electrophoresis analysis of brain extract from PIMT^{-/-} mice identified many proteins that were endogenous PIMT substrates (37). A spot matching the isoelectric point and molecular weight of the Hu proteins was present in this analysis, further supporting the notion that the Hu proteins undergo this modification in vivo.

An environment in which cellular stresses are high can cause the release of cellular products that promote the modification of amino acid residues (59-63), including isoaspartylation. As a result, neo-antigens form that can be taken up by antigen-processing cells, presented to T cells, break immune tolerance, and initiate an immune response to a protein that would otherwise be ignored (64). Epitope spreading may subsequently lead to a diversified response as is found in a number of autoimmune diseases (24). Thus, autoantibodies could develop from exposure of the immune system to isoaspartyl forms of self-peptides (22,25). It is reasonable to speculate that isoaspartylated proteins on the surface of dying tumor cells or shed from dying tumor cells may not be properly recognized and/or repaired by PIMT, potentially leading to an immune response. It is noteworthy that areas of necrosis are often present in SCLC tumors, and even in tissue culture, SCLC cells grow in suspension, forming dense suspended clusters (65) that may be poorly oxygenated. In SCLC tumors from human patients and SCLC-prone mice, isoaspartyl conversion of Hu was highest in pathologically confirmed areas of necrosis in the SCLC tumors (FIGS. 7A and B). PIMT activity is necessary to limit accumulation of damaged proteins within cells; to carry out is repair function, it must not only be present in sufficient amounts, but it must also have physical access to the modified protein. From our observations, the enzyme appears to be variably expressed in SCLC tumors, and thus, it may be possible that isoaspartyl-converted proteins are not always repaired in this context.

Even if PIMT is present, this does not guarantee repair of all isoaspartyl residues. For example, it was observed that an unidentified 18-20 kDa protein present in PC12 cells accumulated high levels of isoaspartate, but the level of isoaspartate did not change when PIMT activity was inhibited. It has been suggested that this protein may have been sequestered in the cell in such a way that it was not accessible to PIMT (59). Indeed the higher mobility band we observe with the anti-isoaspartyl Hu antiserum is not very strong when proteins are labeled with PIMT and 3H-SAM. An inability of PIMT to gain access to a protein due to its location or aggregation (46) could explain why a protein would be enriched in isoaspartyl sites, even if PIMT is expressed. These additional possibilities must be explored further.

Recently, it was shown that the melanocyte differentiation antigen TRP-2 (tyrosinase-related protein-2) undergoes isoaspartyl conversion, resulting in the generation of an anti-isoaspartyl T cell response in vitro and in vivo in which CD8+ T cells are recruited to the tumor site, and autoantibodies capable of binding melanoma cells are produced. Of interest, tumor growth was delayed upon isoaspartyl-TRP-2 immunization, perhaps influencing immunologic clearance of the tumor (66). This brings to mind the observation by some laboratories that anti-Hu autoantibodies in SCLC patients are correlated with comparatively indolent tumor growth relative to antibody negative patients (67-69). These observations emphasize the potential clinical importance of harnessing the immune response to fight cancer and make a strong case for the need to further elucidate the basis of autoimmunity in SCLC.

In conclusion, the present invention has discovered that Hu proteins undergo isoaspartyl conversion in the context of SCLC (FIGS. 7 and 8). While not intending to be bound by any particular theory, based on our observations, we propose that, depending on local conditions in the SCLC tumor, in some patients, PIMT would fail to repair isoaspartylated Hu proteins, possibly leading to the generation of autoantibodies and anti-Hu response (48-52). With the demonstration that the SCLC-associated autoantigen Hu is prone to a highly immunogenic post-translational modification, we provide a tantalizing possibility to explain the mechanism by which the anti-Hu autoimmune response might arise in the context of SCLC. This discovery provides a basis upon which both SCLC detection and therapy are devised.

While general applicability is assumed for the present invention, those skilled in the art will readily recognize that certain proteins may fall outside of this norm due to their variable stabilities. However, such instances may be readily identified with a routine test to verify the suitability of this method and shall not subtract from the general applicability of the present invention.

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

### REFERENCES

The following references are cited herein. The entire disclosure of each reference is relied upon and incorporated herein by reference:
1. Ries, L. A. G., Melbert, D., and Krapcho, M. (2007) SEER Cancer Statistics Review, 1975-2004. National Cancer Institute
2. Schiller, J. H., and Jones, J. C. (1993) Curr Opin Oncol 5, 335-342
3. Henson, R. A., and Urich, H. (1982) Cancer in the nervous system, Blackwell Scientific, Oxford, England
4. Horwich, M. S., Cho, L., Porro, R. S., and Posner, J. B. (1977) Ann Neurol 2, 7-19
5. Szabo, A., Dalmau, J., Manley, G., Rosenfeld, M., Wong, E., Henson, J., Posner, J. B., and Furneaux, H. M. (1991) Cell 67, 325-333
6. Good, P. J. (1995) Proc Natl Acad Sci USA 92, 4557-4561
7. Graus, F., Cordon-Cardo, C., and Posner, J. B. (1985) Neurology 35, 538-543
8. Johnson, B. A., Shirokawa, J. M., Hancock, W. S., Spellman, M. W., Basa, L. J., and Aswad, D. W. (1989) J Biol Chem 264, 14262-14271
9. Potter, S. M., Henzel, W. J., and Aswad, D. W. (1993) Protein Sci 2, 1648-1663
10. Paranandi, M. V., Guzzetta, A. W., Hancock, W. S., and Aswad, D. W. (1994) J Biol Chem 269, 243-253
11. Stephenson, R. C., and Clarke, S. (1989) J Biol Chem 264, 6164-6170
12. Patel, K., and Borchardt, R. T. (1990) Pharm Res 7, 787-793
13. Tyler-Cross, R., and Schirch, V. (1991) J Biol Chem 266, 22549-22556
14. Johnson, B. A., Murray, E. D., Jr., Clarke, S., Glass, D. B., and Aswad, D. W. (1987) J Biol Chem 262, 5622-5629
15. Diliberto, E. J., Jr., and Axelrod, J. (1976) J Neurochem 26, 1159-1165
16. O'Connor, C. M., and Clarke, S. (1985) Biochem Biophys Res Commun 132, 1144-1150
17. McFadden, P. N., and Clarke, S. (1987) Proc Natl Acad Sci USA 84, 2595-2599
18. Mizobuchi, M., Murao, K., Takeda, R., and Kakimoto, Y. (1994) J Neurochem 62, 322-328
19. Billingsley, M. L., Kim, S., and Kuhn, D. M. (1985) Neuroscience 15, 159-171
20. Aswad, D. W. (1984) J Biol Chem 259, 10714-10721
21. Murray, E. D., Jr., and Clarke, S. (1984) J Biol Chem 259, 10722-10732
22. Mamula, M. J., Gee, R. J., Elliott, J. I., Sette, A., Southwood, S., Jones, P. J., and Blier, P. R. (1999) J Biol Chem 274, 22321-22327
23. Young, A. L., Carter, W. G., Doyle, H. A., Mamula, M. J., and Aswad, D. W. (2001) J Biol Chem 276, 37161-37165
24. Mamula, M. J. (1998) Immunol Rev 164, 231-239
25. Doyle, H. A., Gee, R. J., and Mamula, M. J. (2003) J Immunol 171, 2840-2847
26. Yang, M. L., Doyle, H. A., Gee, R. J., Lowenson, J. D., Clarke, S., Lawson, B. R., Aswad, D. W., and Mamula, M. J. (2006) J Immunol 177, 4541-4549
27. Kim, E., Lowenson, J. D., MacLaren, D. C., Clarke, S., and Young, S. G. (1997) Proc Natl Acad Sci USA 94, 6132-6137
28. Yamamoto, A., Takagi, H., Kitamura, D., Tatsuoka, H., Nakano, H., Kawano, H., Kuroyanagi, H., Yahagi, Y., Kobayashi, S., Koizumi, K., Sakai, T., Saito, K., Chiba, T., Kawamura, K., Suzuki, K., Watanabe, T., Mori, H., and Shirasawa, T. (1998) J Neurosci 18, 2063-2074
29. Hardin, J. A., and Thomas, J. O. (1983) Proc Natl Acad Sci USA 80, 7410-7414
30. Monestier, M., Decker, P., Briand, J. P., Gabriel, J. L., and Muller, S. (2000) J Biol Chem 275, 13558-13563
31. Monestier, M., and Kotzin, B. L. (1992) Rheum Dis Clin North Am 18, 415-436
32. Manley, G. T., Smitt, P. S., Dalmau, J., and Posner, J. B. (1995) Ann Neurol 38, 102-110
33. Sillevis Smitt, P., Manley, G., Dalmau, J., and Posner, J. (1996) J Neuroimmunol 71, 199-206
34. Sodeyama, N., Ishida, K., Jaeckle, K. A., Zhang, L., Azuma, A., Yamada, M., Mizusawa, H., and Wada, Y. (1999) J Neurol Neurosurg Psychiatry 66, 97-99
35. Kazarian, M., Calbo, J., Proost, N., Carpenter, C. L., Berns, A., and Laird-Offringa, I. A. (2009) J Neuroimmunol 217, 38-45
36. Park, S., Myszka, D. G., Yu, M., Littler, S. J., and Laird-Offringa, I. A. (2000) Mol Cell Biol 20, 4765-4772
37. Zhu, J. X., Doyle, H. A., Mamula, M. J., and Aswad, D. W. (2006) J Biol Chem 281, 33802-33813
38. Reissner, K. J., Paranandi, M. V., Luc, T. M., Doyle, H. A., Mamula, M. J., Lowenson, J. D., and Aswad, D. W. (2006) J Biol Chem 281, 8389-8398
39. Laskey, R. A., and Mills, A. D. (1975) Eur J Biochem 56, 335-341
40. Chamberlain, J. P. (1979) Anal Biochem 98, 132-135
41. Paranandi, M. V., and Aswad, D. W. (1995) Biochem Biophys Res Commun 212, 442-448
42. Aswad, D. W., and Deight, E. A. (1983) J Neurochem 40, 1718-1726
43. Towbin, H., Staehelin, T., and Gordon, J. (1979) Proc Natl Acad Sci USA 76, 4350-4354
44. Meuwissen, R., Linn, S. C., Linnoila, R. I., Zevenhoven, J., Mooi, W. J., and Berns, A. (2003) Cancer Cell 4, 181-189
45. Johnson, B. A., and Aswad, D. W. (1995) Deamidation and Isoaspartate Formation in Peptides and Proteins, CRC Press, Boca Raton, Fla.
46. Shimizu, T., Fukuda, H., Murayama, S., Izumiyama, N., and Shirasawa, T. (2002) J Neurosci Res 70, 451-461
47. Lowenson, J. D., Kim, E., Young, S. G., and Clarke, S. (2001) J Biol Chem 276, 20695-20702
48. Dalmau, J., Furneaux, H. M., Gralla, R. J., Kris, M. G., and Posner, J. B. (1990) Ann Neurol 27, 544-552
49. Dalmau, J. O., and Posner, J. B. (1997) Semin Oncol 24, 318-328
50. Monstad, S. E., Drivsholm, L., Storstein, A., Aarseth, J. H., Haugen, M., Lang, B., Vincent, A., and Vedeler, C. A. (2004) J Clin Oncol 22, 795-800
51. Verschuuren, J. J., Perquin, M., ten Velde, G., De Baets, M., Vriesman, P. B., and Twijnstra, A. (1999) J Neurol Neurosurg Psychiatry 67, 353-357
52. Tsou, J. A., Kazarian, M., Patel, A., Galler, J. S., Laird-Offringa, I. A., Carpenter, C. L., and London, S. J. (2009) Cancer Detect Prev 32, 292-299
53. Meuwissen, R., Linn, S. C., van der Valk, M., Mooi, W. J., and Berns, A. (2001) Oncogene 20, 6551-6558
54. Dalmau, J., Graus, F., Cheung, N. K., Rosenblum, M. K., Ho, A., Canete, A., Delattre, J. Y., Thompson, S. J., and Posner, J. B. (1995) Cancer 75, 99-109
55. Bidinosti, M., Martineau, Y., Frank, F., and Sonenberg, N. (2010) J Biol Chem 285, 19402-19408
56. Bidinosti, M., Ran, I., Sanchez-Carbente, M. R., Martineau, Y., Gingras, A. C., Gkogkas, C., Raught, B., Bramham, C. R., Sossin, W. S., Costa-Mattioli, M., DesGroseillers, L., Lacaille, J. C., and Sonenberg, N. (2010) Mol Cell 37, 797-808
57. Wang, X., and Tanaka Hall, T. M. (2001) Nat Struct Biol 8, 141-145
58. Vigneswara, V., Lowenson, J. D., Powell, C. D., Thakur, M., Bailey, K., Clarke, S., Ray, D. E., and Carter, W. G. (2006) J Biol Chem 281, 32619-32629
59. Najbauer, J., Orpiszewski, J., and Aswad, D. W. (1996) Biochemistry 35, 5183-5190
60. Clarke, S. (1985) Annu Rev Biochem 54, 479-506
61. Koren, E., Koscec, M., Wolfson-Reichlin, M., Ebling, F. M., Tsao, B., Hahn, B. H., and Reichlin, M. (1995) J Immunol 154, 4857-4864
62. Chazin, W. J., Kordel, J., Thulin, E., Hofmann, T., Drakenberg, T., and Forsen, S. (1989) Biochemistry 28, 8646-8653
63. Galletti, P., Ingrosso, D., Manna, C., Clemente, G., and Zappia, V. (1995) Biochem J 306 (Pt 2), 313-325
64. Doyle, H. A., and Mamula, M. J. (2001) Trends Immunol 22, 443-449
65. Carney, D. N., Gazdar, A. F., Bepler, G., Guccion, J. G., Marangos, P. J., Moody, T. W., Zweig, M. H., and Minna, J. D. (1985) Cancer Res 45, 2913-2923
66. Doyle, H. A., Zhou, J., Wolff, M. J., Harvey, B. P., Roman, R. M., Gee, R. J., Koski, R. A., and Mamula, M. J. (2006) J Biol Chem 281, 32676-32683
67. Dalmau, J., Graus, F., Rosenblum, M. K., and Posner, J. B. (1992) Medicine (Baltimore) 71, 59-72
68. Graus, F., Dalmou, J., Rene, R., Tora, M., Malats, N., Verschuuren, J. J., Cardenal, F., Vinolas, N., Garcia del Muro, J., Vadell, C., Mason, W. P., Rosell, R., Posner, J. B., and Real, F. X. (1997) J Clin Oncol 15, 2866-2872
69. Darnell, R. B., and DeAngelis, L. M. (1993) Lancet 341, 21-22
70. Humphrey et al. Ann Intern Med. Published online 30 Jul. 2013 doi:10.7326/0003-4819-159-6-201309170-00690

1-6. (canceled)

7. An isoaspartyl antigen recognition agent capable of specifically binding to an isoaspartylated protein or an antigenic peptide fragment thereof, wherein:
said antigenic peptide fragment is a peptide between 5 to 40 residues long encompassing a portion of the isoaspartylated protein that includes at least one isoaspartylated residue; and
said isoaspartyl antigen recognition agent is selected from the group consisting of a protein, an antibody, a RNA aptamer, and a small molecule.

8. The isoaspartyl antigen recognition agent of claim 7, wherein said agent is an antibody.

9. The isoaspartyl antigen recognition agent of claim 7, wherein said isoaspartyl protein is selected from the group consisting of HuB, HuC, HuD, Sox1, Sox2, Sox21, ZIC2, ZIC4, Nova-1, voltage-gated calcium or potassium channels (VGCC, VGKC), GABA receptors, nicotinic acetylcholine receptors, neuron-specific enolase, recoverin, synaptotagmin, and synaptophysin.

10. The isoaspartyl antigen recognition agent of claim 9, wherein said antigen recognition agent is an antibody.

11. The isoaspartyl antigen recognition agent of claim 10, wherein said antibody is selected from a humanized antibody, a rabbit antibody, or a mouse antibody.

12. The isoaspartyl antigen recognition agent of claim 7, wherein said isoaspartyl protein is HuD with isoaspartate residues at located at N7, N15, D36, or a combination thereof.

13. The isoaspartyl antigen recognition agent of claim 7, wherein said antigenic peptide fragment is a peptide having the sequence SEQ ID. No. 1.

14. An antibody that specifically binds to a Hu protein containing one or more iso-aspartate residue(s).

15. The antibody of claim 14, wherein said Hu protein is HuD.

16. The antibody of claim 15, wherein said one or more isoaspartate residue(s) are located at position N7, N15, or both.

17. The antibody of claim 14, wherein said antibody is either a monoclonal antibody or a polyclonal antibody.

18. The antibody of claim 14, wherein said antibody is humanized.

19. The antibody of claim 14, wherein said antibody is affinity purified against an affinity column carrying an isoaspartate-containing HuD protein or an antigenic peptide fragment thereof.

20-24. (canceled)

25. A pharmaceutical composition useful for treating small cell lung cancer, comprising;
an effective amount of an antibody that binds specifically to an isoaspartylated neuronal protein ectopically expressed in lung cancer cells; and
a pharmaceutically acceptable carrier,
wherein said neuronal protein is selected from the group consisting of HuB, HuC, HuD, Sox1, Sox2, Sox21, ZIC2, ZIC4, Nova-1, voltage-gated calcium or potassium channels (VGCC, VGKC), GABA receptors, nicotinic acetylcholine receptors, neuron-specific enolase, recoverin, synaptotagmin, and synaptophysin.

26. The pharmaceutical composition of claim 25, wherein said antibody is a monoclonal antibody.

27. The pharmaceutical composition of claim 25, wherein said monoclonal antibody is humanized.

28. The pharmaceutical composition of claim 25, wherein said antibody is a polyclonal antibody.

29. The pharmaceutical composition of claim 25, wherein said antibody is one that specifically binds to an antigenic peptide fragment having the sequence: SEQ ID No. 1.

30-33. (canceled)
